SEQUENCE LISTING (18 sequences)

SEQ ID NO: 1 - protein, 27 aa, H. sapiens
RGDLKHLSKVKKIPSDFEGSGYTDLQE

SEQ ID NO: 2 - protein, 24 aa, H. sapiens
RGDLSKVKKIPSDFEGSGYTDLQE

SEQ ID NO: 3 - protein, 21 aa, H. sapiens
RGDVKKIPSDFEGSGYTDLQE

SEQ ID NO: 4 - protein, 28 aa, H. sapiens
DSQAQKSPVKSKSTHRIQHNIDYLKRGD

SEQ ID NO: 5 - protein, 18 aa, H. sapiens
RGDIPSDFEGSGYTDLQE

SEQ ID NO: 6 - protein, 25 aa, H. sapiens
DSQAQKSPVKSKSTHRIQHNIDRGD

SEQ ID NO: 7 - protein, 15 aa, H. sapiens
RGDDFEGSGYTDLQE

SEQ ID NO: 8 - protein, 19 aa, H. sapiens
DSQAQKSPVKSKSTHRRGD

SEQ ID NO: 9 - protein, 25 aa, H. sapiens
PFKDIPGKGEATGRGDPDLEGKDIQ

SEQ ID NO: 10 - protein, 28 aa, H. sapiens
DIPGKGEATGRGDPDLEGKDIQTGFAGP

SEQ ID NO: 11 - protein, 28 aa, H. sapiens
GKGEATGRGDPDLEGKDIQTGFAGPSEA

SEQ ID NO: 12 - protein, 19 aa, H. sapiens
EATGRGDPDLEGKDIQTGF

SEQ ID NO: 13 - protein, 23 aa, H. sapiens
TDLQERGDNDISPFSGDGQPFKD

SEQ ID NO: 14 - protein, 4 aa, H. sapiens
SGDG

SEQ ID NO: 15 - protein, 4 aa, H. sapiens
  VARIANT 3: Xaa = Any Amino Acid
SGXG

SEQ ID NO: 16 - protein, 12 aa, H. sapiens
DNDISPFSGDGQ

SEQ ID NO: 17 - protein, 12 aa, H. sapiens
  VARIANT 2, 4, 6, 8, 10, 11: Xaa = Any Amino Acid
DXDXSXFXGXXQ

SEQ ID NO: 18 - protein, 12 aa, H. sapiens
DNDISPFSGDGQ

(19) United States
(12) Patent Application Publication
(10) Pub. No.: US 2006/0193916 A1
(43) Pub. Date: Aug. 31, 2006

(54) Formulations of peptides for periodontal and dental treatments

(75) Inventors: Mirella Lazarova, Palo Alto, CA (US); David Rosen, Hayward, CA (US); Yoshinari Kumagai, Emeryville, CA (US)

Correspondence Address: BOZICEVIC, FIELD & FRANCIS LLP, 1900 UNIVERSITY AVENUE, SUITE 200, EAST PALO ALTO, CA, 94303, US

(73) Assignee: Acologix, Inc.

(21) Appl. No.: 11/327,153

(22) Filed: Jan. 5, 2006

Related U.S. Application Data
(60) Provisional application No. 60/642,232, filed on Jan. 7, 2005.

(51) Int. Cl. A61K 38/10; A61K 9/14; A61K 31/728; A61K 33/42

(52) U.S. Cl. 424/486; 514/15; 514/54; 424/603

(57) ABSTRACT

Methods to treat the defects in skeletal tissues characterized by protecting the marrow cells adjacent to the defects from apoptotic and/or necrotic cell death are disclosed. The methods provide additional benefits which are to reduce inflammation and irritation in the marrow tissues and assist promoting new skeletal tissue formation to an application of a simple skeletal formation or regeneration activity such as a bone growth factor. The methods may involve application of pharmacologically active peptidic compounds comprising about 15 to about 28 amino acids in their sequences characterized by containing at least one of an integrin binding motif such as an RGD sequence, a glycosaminoglycan attachment motif, and/or a calcium binding motif. The sequence may be a 23 amino acid sequence formulated for injection, topical application, or dispersed in a matrix such as collagen or a tooth filling composition or gum patch and administered to enhance bone/tooth growth or prevent loss.

### CROSS REFERENCES

This application claims the benefit of U.S. Provisional Application No. 60/642,232, filed Jan. 7, 2005, which application is incorporated herein by reference.

### FIELD OF THE INVENTION

The invention relates generally to the field of bone and dental treatments and more particularly to formulations of peptide sequences for the treatment of bone and teeth.

### BACKGROUND OF THE INVENTION

It is estimated that 90% of the entire population experience dental caries in their lives, and that over 50% of the adults are affected by certain stages of periodontal disease in the United States. The annual cost to treat dental and periodontal problems was approximately $64 billion per year in the United States alone in 2001. The annual cost of the treatment of periodontal disease was approximately $6.5 billion amongst it (Health Care Financing Administration, Department of Health and Human Services).

It is believed that dental caries are caused by acidic condition in the oral cavity. For instance, sugars are converted to acid and dissolve the surface of the teeth. Although only enamel and the outermost region of dentin are affected in many cases, the damage can reach the pulp cavity in severe cases. This can result in significant inflammation and pain. Because dentin contains numerous microtubules that extend from the pulp well into the dentin layer, and these tubules are believed to also contain processes from pulp odontoblasts, cavities that approach the pulp can also result in pain and possible inflammation. In cases where there is only a minimal layer of dentin left to protect the pulp, teeth can become hypersensitive to temperature. The most typical treatment for a “routine” caries lesion (i.e., the dental defect) is to clean the defect and then fill it with a non-degradable material such as metal, alloy, or polymer resin. However, these materials such as metals and unpolymerized residual monomer in the resins can often penetrate into the pulp through the dentin tubules or directly affect the exposed pulp to worsen the inflammation and pain.

In some cases, natural regeneration of dentin over the exposed pulp is attempted. Such newly regenerated dentin over the pulp is called “dentin-bridge.” A dentin-bridge could be formed before the cavity is permanently sealed with the non-degradable materials, or even after the cavity is permanently sealed.

Certain formulations of calcium salts represented by calcium hydroxide [Ca(OH)₂] paste are often used for dentin-bridge formation. This treatment is called “pulp-capping.” In the pulp-capping therapy, a calcium salt formulation is placed over the exposed pulp or on the top of the residual thin dentin layer that covers the pulp. One rationale for using calcium salts is that they are very basic (pH˜9.5 or higher) and thus can cause local irritation in the pulp. This in turn results in the pulp cells producing additional “reactive” dentin. Since the calcium salt is left in the dental defect, it often continues to exert its effect which can sometimes lead to a filling of the pulp cavity with dentin. This excessive dentin can result in pain.

The pulp cavity is located in the central core of tooth and contains the nerve, vascular supply, and also serves as a reservoir of pulp cells. Dental pulp cells are somewhat like marrow cells for a tooth and can differentiate into variety of dental cells such as odontoblasts, ameloblasts, and cementoblasts that ultimately form dentin, enamel, and cement, respectively. Necrosis and apoptosis of the pulp cells as a result of inflammation and irritation caused by the current standard treatments would reduce the number of cells available for the dental tissue regeneration, and, in turn, delay the healing process.

If the pulp is damaged significantly or substantially exposed, it is often removed in a procedure known as a “pulpotomy” or “root canal.” Also, as the pulp cavity accommodates peripheral neurons that reach from the adjacent bone, the pulp inflammation caused by pulp-capping treatment sometimes results in intolerable pain. Pulpotomy is employed to cure such pain. However, once pulp has been removed, the tooth permanently loses its self-healing ability and reduced its biological viability. Therefore, dental specialists always attempt to avoid pulpotomy as much as possible.

Even if the pulp survives after the pulp-capping treatment with calcium salts, the newly generated dentin-bridge by calcium salts is usually not as hard as the normal dentin and called osteodentin.

In summary, the current standard treatment of dental cavities or defects depends on the type and degree of decay. In deep cavities where the pulp capping procedure is needed, sealing materials and/or calcium salts, such as calcium hydroxide are often used. Despite their limited success, such treatments often result in uncontrolled or improper sealing of the pulp cavity which can often lead to future problems such as pain, pulp damage, and ultimate loss of the tooth. Thus, there are needs for a new method to treat dental defects without damaging the pulp and keep the entire tooth tissue vital.

While dental caries and defects widely affect children and adults, periodontal disease affects mostly adults, in particular, the aged. The patient's gum is inflamed and destroyed, and the alveolar bone that supports the teeth is degenerated in periodontal disease. Cement that composes the core of the root is also damaged, and subsequently, teeth fall out.

The enlarged space between a tooth and the supporting bone called “pocket” can be filled with a space-filling or osteoconductive biocompatible material such as bone or dental matrix molecules or a mineral such as β-tricalcium phosphate (β-TCP) to prevent the loss of the tooth. A bone growth factor such as BMP, FGF, or platelet derived growth factor (PDGF) can be used with the space-filling materials to promote the new bone regeneration in the pocket. If the tooth becomes improperly anchored in the surrounding bone then it will be lost. Once removed, the most common treatment or repair is the placement of a dental implant. An artificial implant is placed in the socket where the tooth was lost. In severe cases, an entire denture is replaced by implant. However, because alveolar bone is severely degenerated in these patients, the implants are not necessarily fixed well in the bone and can also become unstable. Augmentation of bone surrounding the area of the lost tooth is needed to ensure a securely placed and functional implant. Similar biocompatible materials to the ones used to fill bony pockets (with or without bone growth factors) can be used to fill the socket before the implant is placed. When such alveolar bone is severely damaged, autogenous bone grafting is sometimes performed. In this case, a bone graft can be taken from surrounding alveolar bone and/or a skeletal tissue in another part of the body, or a “generic” freeze dried allogenic demineralized bone material can be used. However, the cost of this treatment is extremely high and involves highly skilled periodontists and complicated surgical procedures.

Current treatment of periodontal disease involving biocompatible materials, bone growth factors, implants, and surgical procedures does not meet all clinical needs. Therefore, there is significant demand for a novel method to regenerate high quality alveolar bone that can properly support and maintain existing teeth and/or dental implants.

### SUMMARY OF THE INVENTION

Formulations comprised of a 23 amino acid sequence in a pharmaceutically acceptable carrier are disclosed. The carrier may be an osteoconductive matrix particularly adapted to be applied to teeth and/or bone such as β-TCP, HA, collagen, PLA and related polymers.

The teeth may be treated by placing the formulation on the surface of teeth and/or in an opening drilled into the teeth to remove decay. The alveolar bone supporting the teeth may be treated by injecting the formulation into the bone and/or tissue surrounding the bone.

Although the formulation may be repeatedly administered an aspect of the invention is obtaining desired results with a single administration without any subsequent application of a formulation of the invention.

The 23 amino acid sequence comprises the following motifs: an integrin binding motif sequence; a glycosaminoglycan binding motif; and/or a calcium-binding motif. The amino acids may be in the D- or L-conformation. The remaining monomer units (the sequence other than the aforementioned motifs) in the compound may be amino acid analogs. The remaining monomer units are preferably naturally occurring amino acids having a sequence which are substantially the same as an amino acid sequence contiguous with the RGD sequence in the naturally occurring protein, matrix extracellular phosphoglycoprotein (MEPE) (Rowe et. al., Genomics (2000) 67:56-68).

An aspect of the invention is a formulation for and a method of treating a skeletal tissue defect, comprising:

identifying an area of a skeletal tissue defect in a patient; and

administering to the patient an amount of a peptide compound sufficient to reduce inflammation in the area of the skeletal tissue defect, which may be the surface of teeth and/or in an opening drilled into the teeth to remove decay wherein the peptide compound comprises about 18 to about 28 amino acids in a sequence, wherein the peptide compound enhances bone growth, wherein each amino acid may be in D- or L-conformation, and wherein the sequence comprises an integrin binding motif, a glycosaminoglycan binding motif, and/or a calcium binding motif particularly where the calcium binding motif has the sequence DNDISPFSGDGQ (SEQ ID NO:18) by itself or bound to a molecule such as PEG, Fc or the like which improves the half life of the peptide.

Another aspect of the invention is a formulation for and a method of treating a skeletal tissue defect, comprising:

identifying bone marrow cells adjacent to a skeletal tissue defect in a patient; and

administering to the patient an amount of a peptide compound sufficient to reduce bone marrow cell death in the identified area adjacent the skeletal tissue defect, wherein the peptide is dispersed in an osteoconductive matrix and the peptide comprises about 18 to about 28 amino acids in a sequence, wherein the peptide compound enhances bone growth, wherein each amino acid may be in D- or L-conformation, and wherein the sequence comprises an integrin binding motif, a glycosaminoglycan binding motif, and/or a calcium binding motif, particularly wherein the integrin binding motif is an RGD sequence, and wherein the glycosaminoglycan motif has the sequence SGDG (SEQ ID NO:14) and wherein the skeletal tissue may be chosen from alveolar and jaw bone as well as dental tissue.

Formulations for the treatment of periodontal disease and specifically to improve bone growth such as the jaw bone area beneath teeth are disclosed wherein the formulations are comprised of a peptide component wherein the peptide compound comprises about 15 to about 28 amino acids in a sequence, wherein the peptide compound enhances bone growth, wherein each amino acid may be in D- or L-conformation, and wherein the sequence comprises a binding motif chosen from an integrin binding motif, a glycosaminoglycan binding motif, and a calcium binding motif.

The peptide component may have a calcium binding motif having the sequence DXDXSXFXGXXQ (SEQ ID NO:17), wherein X is any amino acid. Specifically, the peptide may have calcium binding motif that has the sequence DNDISPFSGDGQ (SEQ ID NO:18). The peptide compound of the invention may have the peptide sequence TDLQERGDNDISPFSGDGQPFKD (SEQ ID NO:13).

The peptide component may be bound to and/or dispersed in another biocompatible and biodegradable polymer. Such polymers include polyglycolide (PGA), poly(DL-lactide) (DL-PLA), poly(DL-lactide-co-glycolide) (DL-PLG), poly(L-lactide) (L-PLA), poly(L-lactide-co-glycolide) (L-PLG), polycaprolactone (PCL), polyethylene glycol (PEG), polydioxanone, a polyesteramide, a copolyoxalate and a polycarbonate.

The formulation may include the peptide sequence bound to the biocompatible polymer or the sequence by itself. Other biologically active and inactive compounds may be bound to the peptide sequence or they may be used as carrier with a carrier that is used in combination with the peptide sequence. For example, a formulation may include β-TCP which is β-tricalciumphosphate or hyaluronic acid (HA), various forms of collagen and different forms of polylactic acid (PLA). Components such as β-TCP, HA, collagen or PLA may be used a carriers in which the peptide sequence is dispersed. When the peptide sequence is bound to other polymers such as polyethylene glycol to form a pegalated protein or pegalated peptide such a combination can increase the half life of the peptide.

Methods to treat the defects in bone and/or teeth which are characterized by protecting the marrow cells adjacent to the defects from inflammation, necrosis and apoptosis to retain the high biological viability of the local hard tissues are presented. The methods involve direct application of a formulation of a pharmacologically active ingredient which has an activity to protect marrow cells from inflammation, necrosis or apoptosis. The teeth may be treated by applying such a formulation on the surface of teeth and/or in an opening drilled into the teeth to remove decay. The alveolar bone supporting the teeth may be treated by injecting the formulation into the bone and/or tissue surrounding the bone. Other bone tissues may be treated by injecting the formulation into a tissue near a defect. Although the formulation may be repeatedly administered, an aspect of the invention is obtaining desired results with a single administration without any subsequent application of a formulation of the invention.

An aspect of the invention is that the method of the invention enhances dental and periodontal health.

Another aspect of the invention is that the method of the invention increases the number of viable osteoblastic and/or odontoblastic cells in a marrow tissue.

An aspect of the invention is a formulation used in the methods comprising about 15 to about 28 amino acid peptidic sequence and/or its analogs. The peptidic sequence comprises one or more of the following three motifs: an integrin binding motif; a glycosaminoglycan attachment motif; and/or a calcium-binding motif. The amino acids may be in the D- or L-conformation. The remaining monomer units (the sequence other than the aforementioned motifs) in the compound may be amino acid analogs. The remaining monomer units are preferably naturally occurring amino acids having a sequence which are substantially the same as an amino acid sequence contiguous with the RGD sequence in a synthetic peptide, AC-100 (Hayashibara, et. al., Journal of Bone and Mineral Research 19:455-462, 2004), or an amino acid sequence contiguous with the RGD sequence in the naturally occurring protein, matrix extracellular phosphoglycoprotein (MEPE) (Rowe et. al., Genomics 67:56-68, 2000).

Another aspect of the invention is that the formulation of the invention reduces inflammation.

Another aspect of the invention is that the formulation of the invention protects the marrow cells from nectrotic or apoptotic cell death.

Another aspect of the invention is to provide a formulation for therapeutic use which comprises a sufficient concentration of active compound of the invention and can be administered to the pulp of teeth, the space between the root of teeth and gum, or alveolar bone to prevent the damage on teeth and/or alveolar bone in addition to regenerating the hard tissue in the damaged teeth and/or alveolar bone.

Another aspect of the invention is a formulation which may be any of collagen, synthetic polymer resins, calcium salts, hyaluronic acid, polylactic acid, polyethylene glycol, saline, ceramics such as hydroxyapatite or β-tricalcium phosphate (β-TCP), or material used in filling a defect in bone or teeth as a carrier having therein a therapeutically effective amount of the 15 to 28 amino acid peptidic sequence of the invention.

These and other aspects, objects, advantages, and features of the invention will become apparent to those persons skilled in the art upon reading the details of the subject invention, as more fully described below.

### BRIEF DESCRIPTIONS OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 shows a timeline for different groups of the Example 1 described here.

FIG. 2 is a schematic representation of the treatment procedure described in Example 1 described here.

FIGS. 3A and 3B demonstrates dentinogenesis on day 28 by single or multiple applications of AC-100, respectively, in Example 1.

FIG. 4 summarizes the dentinogenesis on day 28 by different therapeutic regimens in Example 1.

FIG. 5 includes 5A and 5B and exhibits tissue reaction on days 3 (5A) and 28 (5B) after initial treatment in different therapeutic regimens Example 1.

FIG. 6 includes 6A and 6B and demonstrates the degree of inflammation in the pulp on days 3 (6A) and 28 (6B) after sealing of the dental cavities in Example 1.

FIG. 7 includes 7A and 7B and shows the number of apoptotic cells in the pulp on days 3 (7A) and 28 9 7B) after sealing of the dental cavities in Example 1.

FIG. 8 is a schematic representation of the study protocol described in Examples 2 described here.

FIG. 9 shows a timeline for different groups of the Example 2 and shows the numbering system for the teeth.

FIG. 10 is a bar graph showing the bone ingrowth after 28 days in the alveolar regeneration study in Example 2.

FIG. 11 shows three bar graphs showing the quality of new bone and defects bridged by new bone, as well as the combined data of these two at 28 days with the alveolar regeneration study in Example 2.

FIG. 12 shows three bar graphs showing results of tissue reaction at 3 days.

FIG. 13 is two bar graphs showing the presence of fibrous tissue at 3 and 28 days.

FIGS. 14A and 14B are bar graphs showing results obtained at 3 days and 28 days respectively in the alveolar regeneration study.

FIGS. 15A and 15B are bar graphs showing results at 3 days and 28 days respectively with respect to inflammation in the alveolar regeneration study.

### DETAILED DESCRIPTION OF THE INVENTION

Before the methodology, peptides, analogs, and formulations of the present invention are described, it is to be understood that this invention is not limited to any particular embodiment described, as such may, of course, vary. It is also to be understood that the terminology used herein is with the purpose of describing particular embodiments only, and is not intended to limit the scope of the present invention which will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited. The present disclosure is controlling to the extent there is a contradiction between the present disclosure and a publication incorporated by reference.

It must be noted that as used herein and in the appended claims, the singular forms “a”, “and”, and “the” include plural referents unless the context clearly dictates otherwise. Thus, for example, reference to “a peptide” includes a plurality of such peptides and reference to “the method” includes reference to one or more methods and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The terms “peptide” and “peptidic compound” are used interchangeably herein to refer to a polymeric form of amino acids of from about 15 to about 28 amino acids, which can comprise coded and non-coded amino acids, chemically or biochemically modified or derivatized amino acids, L- or D-amino acids, peptides having modified peptide backbones, and peptides comprising amino acid analogs. The peptidic compounds may be polymers of: (a) naturally occurring amino acid residues; (b) non-naturally occurring amino acid residues, e.g. N-substituted glycines, amino acid substitutes, etc.; or (c) both naturally occurring and non-naturally occurring amino acid residues/substitutes. In other words, the subject peptidic compounds may be peptides or peptoids. Peptoid compounds and methods for their preparation are described in WO 91/19735, the disclosure of which is herein incorporated by reference. A peptide compound of the invention may comprise 23 amino acids or from 15 to 28 amino acids or from 20 to 26 amino acids. The active amino acid sequence of the invention comprises at least one of three motifs which may be overlapping which are: an integrin binding motif sequence; a glycosaminoglycan attachment motif sequence; and/or a calcium-binding motif.

The terms “treat”, “treating”, “treatment” and the like are used interchangeably herein and mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed the disease. “Treating” as used herein covers treating a disease in a vertebrate and particularly a mammal and most particularly a human, and includes:

(a) preventing the disease from occurring in a subject which may be predisposed to the disease but has not yet been diagnosed as having it;

(b) inhibiting the disease, i.e. arresting its development; or

(c) relieving the disease, i.e. causing regression of the disease.

The invention is particularly directed towards formulations of peptides and their use which make it possible to treat patient's which have experienced or which would be expected to experience loss of dentin from teeth and/or loss of alveolar bone supporting the teeth and thus is particularly directed towards preventing, inhibiting, or relieving the effects of such loss. A subject is “treated” provided the subject experiences a therapeutically detectable and beneficial effect which may be measured based on a variety of different criteria including increased bone growth, increased dentinogenesis, increased bone strength, increased dentin strength, increased dental viability, decreased inflammation, decreased irritation, decreased pain, or other characteristics generally understood by those skilled in the art to be desirable with respect to the treatment of diseases related to bone.

The term “skeletal defect” refers to any situation in which skeletal mass, substance or matrix or any component of the skeleton, such as calcium and phosphate, is decreased or the bone or the tooth is lost, damaged, or weakened such as in terms of its ability to resist being broken.

The term “skeleton” includes both bone and teeth. In the same manner, the term “skeletal” means both bone and teeth.

The terms “subject,” “individual,” “patient,” and “host” are used interchangeably herein and refer to any vertebrate, particularly any mammal and most particularly including human subjects, farm animals, and mammalian pets.

The term “collagen” is used here to include any type, including, but not limited to, types I, II, III, IV, or any combination thereof. Either atelopeptide or telopeptide-containing collagen may be used; however, when collagen from a xenogeneic source, such as bovine collagen, is used, atelopeptide collagen is generally preferred because of its reduced immunogenicity compared to telopeptide-containing collagen.

Collagen that has not been previously crosslinked by methods such as heat, irradiation, or chemical crosslinking agents is preferred for use in the compositions of the invention, although previously crosslinked collagen may be used. Noncrosslinked atelopeptide fibrillar collagen is commercially available from Angiotech Pharmaceuticals, Inc. of Palo Alto, Calif. (through its acquisition of Cohesion Technologies, Inc. in 2003) at collagen concentrations of 35 mg/ml and 65 mg/ml under the trademarks Zyderm® I Collagen and Zyderm II Collagen, respectively. Glutaraldehyde crosslinked atelopeptide fibrillar collagen is commercially available from Angiotech Pharmaceuticals at a collagen concentration of 35 mg/ml under the trademark Zyplast® Collagen.

Although intact collagen is preferred, denatured collagen, commonly known as gelatin, can also be used in the compositions of the invention. Gelatin may have the added benefit of being degradable faster than collagen.

Because of its tacky consistency, nonfibrillar collagen is generally preferred for use in compositions of the invention that are intended for use as bioadhesives. The term “nonfibrillar collagen” refers to any modified or unmodified collagen material that is in substantially nonfibrillar form at pH 7, as indicated by optical clarity of an aqueous suspension of the collagen.

Collagen that is already in nonfibrillar form may be used in the compositions of the invention. As used herein, the term “nonfibrillar collagen” is intended to encompass collagen types that are nonfibrillar in native form, as well as collagens that have been chemically modified such that they are in nonfibrillar form at or around neutral pH. Collagen types that are nonfibrillar (or microfibrillar) in native form include types IV, VI, and VII.

Chemically modified collagens that are in nonfibrillar form at neutral pH include succinylated collagen and methylated collagen, both of which can be prepared according to the methods described in U.S. Pat. No. 4,164,559, issued Aug. 14, 1979, to Miyata et al., which is hereby incorporated by reference in its entirety. Due to its inherent tackiness, methylated collagen is particularly preferred for use in bioadhesive compositions, as disclosed in commonly owned U.S. Pat. No. 5,614,587.

Hyaluronic acid (hereinafter abbreviated ‘HA’) is used here to describe a bio-polymeric material made up of a repeat unit comprising N-acetyl-D-glucosamine and D-glucuronic acid in a linearly repeated connection, which polymer plentifully exists in vitreous humor, synovial fluid, connective tissues etc. The term ‘HA’ means hyaluronic acid and any of its hyaluronate salts. Hyaluronate salts include but are not limited to inorganic salts such as sodium hyaluronate and potassium hyaluronate etc. and organic salts such as tetrabutylammonium hyaluronate etc. A hyaluronate salt of HA according to the present invention is sodium hyaluronate. Hyaluronic acid is a natural component of connective tissue, including the skin. It plays a critical role in providing volume to skin by retaining water. It is a glycosaminoglycan found in lubricating proteoglycans of the synovial fluid, vitreous humors, cartilage, blood vessels, skin and the umbilical cord.

HA derivatives have been developed in diverse uses for prevention of adhesion after surgical operation, correction of facial wrinkles, dermal augmentation, tissue engineering, osteoarthritic viscosupplement etc. HA derivatives may largely be classified by water solubility into water-soluble derivatives and water-insoluble derivatives. In the case of water-insoluble derivatives, manufacturing methods of those may largely be thought in two ways: one is to react HA with a compound having one functional group to combine this compound with linear chain of HA while the other is to react HA with a compound having two or more functional groups to make crosslinked HA.

There have been reported in several literature various examples to have synthesized many crosslinked, water-insoluble HA derivatives by using compounds such as divinylsulfone, bisepoxide, bishalide, formaldehyde, etc. having two functional groups.

There have been reported in U.S. Pat. No. 4,582,865 to have used divinylsulfone in order to crosslink HA and in U.S. Pat. No. 4,713,448 to have carried out crosslinking reaction by using formaldehyde. And there has been reported in PCT Patent Publication WO86/00912 an example to have used compound containing epoxy groups in order to crosslink various polysaccharides containing carboxyl group.

There is a report that water solubility of HA derivatives is decreased if carboxyl group of HA is activated in aqueous solution by using EDC (1-ethyl-3-(3-dimethylaminopropyl)carbodiimide hydrochloride) and then reacted with compounds containing one amine group to form amide bond (see U.S. Pat. No. 4,937,270). And there is an example to have crosslinked HA with various polyanionic polysaccharides using EDC (see U.S. Pat. No. 5,017,229).

Transforming growth factor-β1 (TGF-β1) is used here to describe a growth factor and immunomodulatory cytokine that is secreted from cells and acts through specific binding interactions with a collection of different cell-surface localized receptors. TGF-β1 is the prototype for a large family of secreted polypeptides that includes the three mammalian TGF-β isoforms (TGF-β1, TGF-β2, and TGF-β3), bone morphogenesis proteins (BMPs), activins, and Mullerian inhibitory substance (MIS). More distantly related members of this protein family include murine nodal gene products, Drosophila decapentaplegic complex gene products, and Vg1 from Xenopus.

In general, TGF-β family proteins are homodimers, wherein each functional protein complex includes two identical, associated monomer subunits. The crystal structure of the TGF-β1 homodimer is known (Hinck et al., Biochem., 35:8517-8534, 1996; Qian et al., J. Biol. Chem., 271:30656-30662, 1996). TGF-β is a very compact protein, having four intramolecular disulfide bridges within each subunit, as well as one intermolecular disulfide bridge.

Each monomer of the protein is synthesized as a large (.about.55 kDa) precursor molecule with a long (about 278 residue) N-terminal pro-region and a much shorter (112 residue, 12.5 kDa) C-terminal active domain (the mature region). During the maturation process, two precursor molecules associate with each other; the pro-region is important for proper folding of and proper association between the two active domain monomers. The pro-region of each monomer is proteolytically cleaved from the associated active domain; in most instances however, the pro-region remains associated with the mature TGF-β fragment. The severed pro-region is referred to as the “latency-associated peptide” (LAP). LAP is responsible for blocking the correctly folded TGF-β homodimer so that it does not interact with its receptor. For an excellent discussion of TGF-β synthesis, see Khalil, Micro. Infect., 1:1255-1263, 1999.

TGF-βs and their receptors are expressed in essentially all tissues, and have been found to be important in many cellular processes. These include cell growth and differentiation, immunosuppression, inflammation, and the expression of extracellular matrix proteins. By way of example, in animal models TGF-β has been shown to attenuate the symptoms associated with various diseases and disorders, including rheumatoid arthritis, multiple sclerosis, wound healing, bronchial asthma, and inflammatory bowel disease, and has been used in the clinical setting to enhance wound healing.

TGF-β1 was the first identified member of the TGF-β family, and has been intensely studied for over 20 years. There are some TGF-β1 antibodies available, but their usefulness in a clinical setting is limited at least in part because they often display some degree of cross-reactivity to other TGF-β family proteins (see, e.g., U.S. Pat. No. 5,571,714). In most experiments, TGF-β is iodinated with ¹²⁵I to enable researchers to track the protein. Radioactive iodination is an expensive and hazardous process, and it usually would be inappropriate to use ¹²⁵I labeled proteins for in vivo experimentation, for instance in clinical trials.

Transforming growth factor-β (TGF-β is considered to be a multifunctional cytokine (Sporn and Roberts, Nature (London), 332: 217-219, 1988), and plays a regulatory role in cellular growth, differentiation and extracellular matrix protein synthesis (Madri et al., J Cell Biology, 106: 1375-1384, 1988). TGF-β inhibits the growth of epithelial cells and osteoclast-like cells in vitro (Chenu et al., Proc Natl Acad Sci, 85: 5683-5687, 1988), but it stimulates enchondral ossification and eventually bone formation in vivo (Critchlow et al., Bone, 521-527, 1995; Lind et al., A Orthop Scand, 64(5): 553-556, 1993; and Matsumoto et al., In vivo, 8: 215-220, 1994). TGF-β-induced bone formation is mediated by its stimulation of the subperiosteal pluripotential cells, which eventually differentiate into cartilage-forming cells (Joyce et al., J Cell Biology, 110: 2195-2207, 1990; and Miettinen et al., J Cell Biology, 127-6: 2021-2036, 1994).

The biological effect of TGF-β in orthopedics has been reported (Andrew et al., Calcif Tissue In. 52: 74-78, 1993; Borque et al., Int J Dev Biol., 37:573-579, 1993; Carrington et al., J Cell Biology, 107:1969-1975, 1988; Lind et al., A Orthop Scand. 64(5):553-556, 1993; Matsumoto et al., In vivo, 8:215-220, 1994). In mouse embryos, staining shows that TGF-β is closely associated with tissues derived from the mesenchyme, such as connective tissue, cartilage and bone. In addition to embryologic findings, TGF-β is present at the site of bone formation and cartilage formation. It can also enhance fracture healing in rabbit tibiae. Recently, the therapeutic value of TGF-β has been reported (Critchlow et al., Bone, 521-527, 1995; and Lind et al., A Orthop Scand, 64(5): 553-556, 1993), but its short-term effects and high cost have limited wide clinical application.

### Invention in General

Peptide sequences comprising 18 to 28 amino acids are disclosed. The sequences are characterized by containing an integrin binding motif such as an RGD sequence, a glycosaminoglycan binding motif, and/or a calcium binding motif, and the remainder of amino acids contiguous with the RGD sequence in matrix extracellular phosphoglycoprotein. The sequence may be a 23 amino acid sequence formulated for injection or dispersed in a matrix such as collagen or a tooth filling composition or gum patch and administered to enhance bone/tooth growth or prevent loss.

Individuals suitable for treatment with a method of the invention include individuals believed to be at risk for, bone loss, or a disorder caused by bone loss, or a disorder whose sequelae include bone loss, including, but not limited to, dental caries, osteoporosis, Paget's disease, renal phosphate leakage, renal osteodystrophy, osteomalacia, osteodystrophy resulting from other causes, osteolysis mediated by cancer, fractures, and hyperparathyroidism. Such individuals include older individuals, post-menopausal women, kidney transplant recipients, and individuals having, or being at risk for, any of the aforementioned disorders.

In order to demonstrate the usefulness of the method of the invention, a 23 amino acid peptide having SEQ ID NO:13 referred to here as AC-100 in a saline formulation was applied to the defects in a tooth and alveolar bone. Although AC-100 has been known to promote proliferation and differentiation of bone formation cells, it was for the first time proven that AC-100 also protected the marrow cells such as dental pulp cells from apoptotic and necrotic cell death. It was also demonstrated for the first time that AC-100 reduced inflammation in the tissue including the marrow. As compared to calcium hydroxide that is widely used to treat dental defects and known to inflame and irritate dental pulp cells to have them release various growth factors and cytokines to promote the hard tissue regeneration, AC-100 has demonstrated to accelerate hard tissue regeneration without causing or even reducing the inflammatory reactions in the affected area. Also, AC-100 has demonstrated to form healthy woven bones in a socket in alveolar bone where a tooth was extracted. Combining these comparable experiments involving AC-100 and other compounds, it was concluded that protecting the marrow cells such as bone marrow cells or dental pulp cells provides additional benefits on regeneration of new skeletal tissues such as bone and dentin. The examples show the additional benefits of marrow cell protection from inflammation, necrosis and/or apoptosis on the top of the already-known skeletal tissue formation activities, which suggests the usefulness of therapeutic methods in general that is characterized by protecting the marrow cells from apoptotic or necrotic cell death.

Therapeuric Methods

The invention provides methods to treat the defects in skeletal tissues such as bone and teeth which are characterized by protecting the marrow cells adjacent to the defects from necrotic or apoptotic cell death to retain the high biological viability of the local hard tissues.

The direct effects of the methods are that the marrow cells adjacent to the skeletal defects of interest are protected from necrotic and/or apoptotic cell death. One of the secondary effects or benefits of the methods is that the healing of the skeletal defects is accelerated. It is partly because the precursor cells for bone and dental tissue within the marrow cell population remain healthy and a sufficient size reservoir of the cells that have potentiality to differentiate into and regenerate new hard tissues can be retained. An alternative reason depending upon the type of the hard tissue of interest is that such precursor cells for bone and dental tissue formation are protected from inflammation or irritation caused by the cytokines that are released by the cells in the tissue where the repair process is in process. Such inflammation or irritation are often worsened when the defects are treated with chemicals such as drugs, resins, and metals as they stimulate the cells to release inflammatory or irritant factors.

The data from the Examples of this invention clearly demonstrated that the marrow cell protection in addition to direct skeletal tissue formation resulted in superior healing of the hard tissue defects in that a larger volume of new hard tissues were regenerated and the quality of such regenerated hard tissues were higher as compared to the hard tissue formation alone group. In particular, the former group showed almost complete reduction in inflammation and prevention of apoptotic or necrotic cell death of the marrow cells that is caused by inflammatory events associated with the healing process while it also demonstrated superior hard tissue regeneration. It was a surprising observation because the tissue healing process usually involves certain inflammatory events in the tissue that stimulate the cells to release the factors to repair the tissue.

The marrow cells protected by the methods in this invention may be either bone marrow or dental pulp cells but not necessarily restricted to them. Any marrow cells, a reservoir of the marrow cells, or a locally populated marrow cells which include a subpopulation of the precursor cells that are capable of differentiating into skeletal tissue formation cells are within the scope of this invention as far as they are located close enough to a hard tissue defect to be used to treat such defect. The skeletal tissue formation cells mean osteoblasts, pre-osteoblasts, stromal cells, fibroblasts, odontoblasts, pre-odontoblasts, ameroblasts, pre-ameroblasts, cementoblasts, pre-cementoblasts, dental pulp cells, chondrocytes, pre-chondrocytes, and their related cells which possess the potency to differentiate into a hard tissue depending upon the internal or external stimulation and influence on the cells.

When bone marrow cells such as stem cells, stromal cells, fibroblasts, pre-osteoblasts, and/or osteoblasts are protected by the method of this invention, the treated defects will be the ones in bone. When dental pulp cells are protected by the method of this invention, the treated defects will be the ones in dental tissues. More specifically, when pre-odontoblasts and/or odontoblasts which typically reside in dental pulp are protected, the treated defects will be the ones in dentin. When pre-ameroblasts and/or ameroblasts are protected, the treated defects will be the ones in enamel. When pre-cementoblasts and/or cementoblasts are protected, the treated defects will be the ones in cement, which is the hard surface tissue on the root of a tooth. When pre-chondrocytes and/or chondrocytes are protected, the treated defects will be the ones in cartilage. Marrow cells are mixed population of different cells in the different lineage. Therefore, more than one subpopulations of different cell types in the different lineage can be the target of the methods of this invention.

The methods may involve direct application of a formulation containing a pharmacologically active compound which has an activity to protect marrow cells from inflammation, necrosis or apoptosis to the hard tissue defects that are to be treated. The teeth may be treated by applying such a formulation on the surface of teeth and/or in an opening drilled into the teeth to remove decay. A local bone tissue such as alveolar bone which supports the teeth may be treated by injecting the formulation into the bone and/or tissue surrounding the bone. Other bone tissues may be treated locally by injecting the formulation into the bone or a tissue near a defect. Although the formulation may be repeatedly administered, an aspect of the invention is obtaining desired results with a single administration without any subsequent application of a formulation of the invention.

Peptidic Compounds

In a preferred embodiment of this invention, the formulation contains an effective amount of a peptidic compound.

The peptidic compound of the invention is a peptide comprising from about 15 to about 28 amino acids. The amino acids are preferably one of the twenty naturally occurring L-amino acids. However, D-amino acids may be present as may amino acid analogs. A peptide of the invention will comprise one of three of the following amino acid sequence motifs: an integrin binding motif such as RGD sequence; a glycosaminoglycan attachment motif; and/or a calcium binding motif. Individual amino acids may be present in the peptides in either the L or the D isoform, but preferably in the L form. A peptide of the invention can be amidated or non-amidated on its C-terminus, or carboxylated or non-carboxylated on its N-terminus. The peptide of the invention will contain a glycosaminoglycan attachment motif such as SGDG (SEQ ID NO:14) sequence in L- or D-isomer form. A compound of the invention is still further characterized by biological activity i.e. it reduces inflammation in a defected tissue under healing process and protects apoptotic or necrotic cell death that are typical to such an healing process.

Specific examples of peptides of the invention which comprise the RGD sequence as the terminal sequence include the following:

| | |
|---|---|
| RGDLKHLSKVKKIPSDFEGSGYTDLQE | (SEQ ID NO:1) |
| RGDLSKVKKIPSDFEGSGYTDLQE | (SEQ ID NO:2) |
| RGDVKKLPSDFEGSGYTDLQE | (SEQ ID NO:3) |
| DSQAQKSPVKSKSTHRIQHNLDYLKRGD | (SEQ ID NO:4) |
| RGDIPSDFEGSGYTDLQE | (SEQ ID NO:5) |
| DSQAQKSPVKSKSTHRIQHNIDRGD | (SEQ ID NO:6) |
| RGDDFEGSGYTDLQE | (SEQ ID NO:7) |
| DSQAQKSPVKSKSTHRRGD | (SEQ ID NO:8) |

Specific examples of the peptides of the invention which comprise the RGD internally include the following:

| | |
|---|---|
| PFKDIPGKGEATG RGDPDLEGKDIQ | (SEQ ID NO:9) |
| DIPGKGEATG RGDPDLEGKDIQTGFAGP | (SEQ ID NO:10) |
| GKGEATG RGDPDLEGKDIQTGFAGPSEA | (SEQ ID NO:11) |
| EATG RGDPDLEGKDIQTGF | (SEQ ID NO:12) |

A peptide of the invention comprises a glycosaminoglycan attachment motif. A glycosaminoglycan attachment motif has the consensus sequence SGXG (SEQ ID NO:15), wherein X is any amino acid. In some embodiments, a glycosaminoglycan attachment motif has the sequence SGDG (SEQ ID NO:14).

A peptide of the invention also comprises a calcium binding motif. In some embodiments, a calcium binding motif has the sequence DNDISPFSGDGQ (SEQ ID NO:16). Also included in the term “calcium binding motif” are amino acid sequences that differ from SEQ ID NO:16 by one, two, three, four, five, six, seven, or eight amino acids. Of particular interest in many embodiments are motifs that conserve amino acids 1, 3, 5, 7, 9, and 12 of SEQ ID NO:16. Thus, in some embodiments, a peptide of the invention comprises, as a calcium-binding motif, the sequence DXDXSXFXGXXQ (SEQ ID NO:17), wherein X is any amino acid or amino acid analog.

In other embodiments, a calcium binding motif has the sequence DX₁X₂X₃X₄X₅X₆X₇X₈X₉X₁₀X₁₁X₁₂, wherein:

X₁ is any amino acid;

X₂ is D, N, or S;

X₃ is I, L, V, F, Y, or W;

X₄ is D, E, N, S, T, or G;

X₅ is D, N, Q, G, H, R, or K;

X₆ is G or P;

X₇ is L, I, V, M, C;

X₈ is D, E, N, Q, S, T, A, G, or C;

each of X₉ and X₁₀ is independently any amino acid;

X₁₁ is D or E; and

X₁₂ is L, I, V, M, F, Y, or W.

In other embodiments, a calcium binding motif has the sequence X₁X₂X₃X₄C(X₅)ₙC(X₆)ₘCX₇X₈X₉X₁₀X₁₁X₁₂X₁₃X₁₄C, wherein

each of X₁, X₃, and X₄ is independently D, E, Q, or N;

each of X₂, X₅, X₆, X₇, X₉, X₁₀, X₁₁, X₁₂, and X₁₄ is independently any amino acid;

n is 3-14;

m is 3-7;

X₈ is D or N; and

X₁₃ is F or Y.

In other embodiments, a calcium binding motif has the sequence X₁X₂X₃X₄X₅DX₆X₇X₈X₉X₁₀X₁₂X₁₃X₁₄X₁₅X₁₆X₁₇X₁₈X₁₉X₂₀X₂₁, wherein

each of X₁ and X₂ is independently L, I, V, M, F, Y, or W;

each of X₃, X₄, X₆, X₇, X₈, X₁₀, X₁₁, X₁₂X₁₅, X₁₈, and X₁₉ is independently any amino acid;

X₅ is L or K;

X₉ is D or N;

X₁₃ is D, N, S, or G;

X₁₄ is F or Y;

X₁₆ is E or S; X₁₇ is F, Y, V, or C;

X₂₀ is L, I, V, M, F, or S; and X₂₁ is L, I, V, M, or F.

In other embodiments, a calcium binding motif has the sequence DX₁X₂X₃X₄X₅X₆GX₇DX₈X₉X₁₀GGX₁₁X₁₂X₁₃D, wherein

each of X₁, X₃, X₄, X₅, X₆, X₇, X₈, X₁₀, X₁₁, X₁₂, and X₁₃ is independently any amino acid; and

each of X₂ and X₉ is independently L or I.

Calcium binding motifs are known in the art and have been described amply. See, for example, Springer et al. (2000) Cell 102:275-277; Kawasaki and Kretsinger (1995) Protein Prof. 2:305-490; Moncrief et al. (1990 J. Mol. Evol. 30-522-562; Chauvaux et al. (1990) Biochem. J. 265:261-265; Bairoch and Cox (1990) FEBS Lett. 269:454-456; Davis (1990) New Biol. 2:410-419; Schaefer et al. (1995) Genomics 25:638-643; and Economou et al. (1990) EMBO J. 9:349-354. Any known calcium binding motif can be included in a peptidic compound of the invention.

A peptide of the invention comprises at least one of an integrin binding motif, a glycosaminoglycan binding motif, and/or a calcium binding motif. The motifs may be present in the peptide in any order relative to one another. The motifs may be separated from one another by one, two, three, four, five, six, seven, eight, nine, or ten amino acids, or more. Furthermore, a motif may overlap with one or more other motifs. As one non-limiting example, a peptide having the sequence TDLQERGDNDISPFSGDGQPFKD (SEQ ID NO:13) comprises all three motifs, which overlap with one another. This peptide is referred here as AC-100.

All or any of the amino acids in the above sequences may be in the D- or L-conformation and may be substituted with equivalent analogs. The preferred embodiments comprise naturally occurring amino acids in the L-conformation.

All or any of the above sequences may be amidated, non-amidated, or otherwise modifed on their C-terminus, or carboxylated, non-carboxylated, or otherwise modified on their N-terminus.

In addition, multimers of any of the foregoing peptides are provided. Multimers include dimers, trimers, tetramers, pentamers, hexamers, etc. Thus, a peptide of the invention having a length of from about 10 to about 50 amino acids can be multimerized, optionally with an intervening linker, such that a subject peptide occurs in tandem arrays of two, three, four, five, six, or more copies. Furthermore, two or more different peptides of the invention can be multimerized with one another, forming “heteromultimers.” Thus, e.g., a multimer may comprise a first and a second peptide, linked together by peptide bonds, optionally with a linker molecule such as one to ten glycine residues.

Peptidic compounds of the invention can be obtained using any known method, including, e.g., solid phase peptide synthesis techniques, where such techniques are known to those of skill in the art. Methods for synthesizing peptides are well known in the art and have been amply described in numerous publications, including, e.g., “The Practice of Peptide Synthesis” M. Bodanszky and A. Bodanszky, eds. (1994) Springer-Verlag; and Jones, The Chemical Synthesis of Peptides (Clarendon Press, Oxford)(1994). Generally, in such methods a peptide is produced through the sequential additional of activated monomeric units to a solid phase bound growing peptide chain. Also of interest is the use of submonomers in solid phase synthesis, as described in WO 94/06451, the disclosure of which is herein incorporated by reference.

Instead of solid phase synthesis, the subject peptidic compounds of the subject invention may be prepared through expression of an expression system comprising a polynucleotide encoding the peptidic compound. Any convenient methodology may be employed, where methodologies that may be employed typically include preparation of a nucleic acid molecule comprising a nucleotide sequence encoding the subject peptide, introduction of the encoding region into a vector for expression, transformation of a host cell with the vector, and expression and recovery of the product. Protocols for accomplishing each of the above steps are well known in art. See Sambrook, Fritsch & Maniatis, Molecular Cloning, A Laboratory Manual (Cold Spring Harbor Press, Inc.)(1989).

AC-100, a 23 amino acid linear peptide indicated as SEQ ID No:13 herein was originally discovered as a specific bone formation motif in a large molecule of 525 amino acid named matrix extracellular phosphoglycoprotein (MEPE) (see PCT Pat. Appl. PCT/US01/25542 for AC-100; see Rowe, et. al., Genomics 67:56-68, 2000, U.S. Pat. No. 6,818,745, and U.S. Pat. No. 6,673,900 for MEPE). AC-100 has thus far demonstrated potent activities on proliferation and differentiation of osteoblastic cells that were comparable to those of some growth factors such as BMP and IGF-1 (Nagel, et. al., Journal of Cellular Biochemistry 93:1107-1114, 2004). AC-100 has also demonstrated potent activities on proliferation but not differentiation of human dental pulp cells (Liu, et. al., Journal of Dental Research 83:496-499, 2004). AC-100 and some of its analogue peptides have further shown in vitro and in vivo bone formation activities in mice that were comparable to the same activities of fibroblast growth factor (FGF) (Hayashibara, et. al., Journal of Bone and Mineral Research 19:455-462, 2004). On the other hand, MEPE has demonstrated traditionally hypothesized phosphatonin activity that is to regulate serum levels of phosphate (Rowe, et. al., Bone, 2004). Nothing in these prior arts related to MEPE, AC-100, or their orthologues or analogues has suggested that AC-100 or its related molecules possess protecting activities on any cells. Nothing in the arts suggested anti-inflammatory activities of these molecules, either. AC-100 was particularly characterized with a few characteristic motifs such as integrin binding motif and glycosaminoglycan attachment motif but they were proven to be related to its bone formation activities only (Hayashibara, et. al., Journal of Bone and Mineral Research 19:455-462, 2004). It was surprising that a peptide like AC-100 possessed protecting activities on the cells from apoptotic and necrotic cell death as well as reduction of inflammation.

Another surprising fact was that a peptide with an integrin binding motif, in particular, RGD sequence, has demonstrated cell protecting activities. It has been reported that a synthetic peptide containing the RGD sequence inhibited bone formation and resorption in a mineralizing organ culture system of fetal rat skeleton (Gronowicz et. al. Journal of Bone and Mineral Research 9(2):193-201 (1994)).

As used herein, an “effective amount” of a peptidic compound of the invention is an amount that reduces inflammation, and/or protects the cells from their apoptotic or necrotic cell death by at least about 10%, at least about 15%, at least about 20%, at least about 25%, at least about 30%, at least about 35%, at least about 40%, at least about 45%, at least about 50%, at least about 55%, or at least about 60%, or more, when compared to a suitable control. Suitable controls are, in the case of experimental animals, an animal not treated with the peptide, e.g., treated with vehicle, or treated with an irrelevant peptide; and in the case of human subjects, a human subject treated with a placebo, or a human subject before treatment with a peptide of the invention.

In some embodiments, an effective amount of a peptidic compound of the invention is an amount that reduces inflammatory reaction of the tissues in and around the ones involved in a healing process, and therefore reduces the number of cells die from necrosis or apoptosis, by at least about 10%, at least about 15%, at least about 20%, at least about 25%, at least about 30%, at least about 35%, at least about 40%, at least about 45%, at least about 50%, at least about 55%, or at least about 60%, or more, when compared to a suitable control.

In another embodiments, an effective amount of a peptidic compound of the invention is an amount that reduces pain in or near the tissues in a healing process by at least about 10%, at least about 15%, at least about 20%, at least about 25%, at least about 30%, at least about 35%, at least about 40%, at least about 45%, at least about 50%, at least about 55%, or at least about 60%, or more, when compared to a suitable control.

Whether a given peptide reduces inflammation, and/or necrotic cell death, and/or apoptotic cell death in an individual can be determined using any known assay to measure any known parameter associated with any one or more of reduced production of biological inflammatory compounds such as lymphokines or cytokines, reduced pain by appropriate clinical pain scores, increased bone and/or dental viability, and the like. Such methods are standard in the art.

Individuals suitable for treatment with the methods of the invention are individuals having a defect in skeletal tissues such as bone and teeth, including, but not limited to, dental caries, periodontal disease, tooth extraction, loss of teeth, loss or loosened dental implant, osteoporosis, Paget's disease, renal osteodystrophy, osteomalacia, resulting from other causes, osteolysis mediated by cancer, fractures, hyperparathyroidism, and dental decay. Such individuals include older individuals, post-menopausal women, kidney transplant recipients, and individuals having, or being at risk for, any of the aforementioned disorders.

Methods of Application

Peptidic compounds of the invention are applied to an individual using any available local administration method and route suitable for drug delivery, including in vivo and ex vivo methods.

Conventional and pharmaceutically acceptable methods of local application include intramuscular, subcutaneous, intradermal, topical application, and other parenteral or topical methods of administration. Peptidic compounds of the invention can be administered in a single dose or in multiple doses.

Peptidic compounds of the invention can be administered locally to a subject using any available conventional methods and routes suitable for delivery of conventional drugs.

Parenteral routes of local administration include, but are not necessarily limited to, topical, transmucosal, transdermal, subcutaneous, intramuscular, intracapsular, intraspinal, and intrasternal routes. Parenteral application should be carried to effect local delivery of peptides of the invention.

Typically, a peptidic compound of the invention is formulated with a pharmaceutically acceptable excipient for delivery to an individual in need thereof.

Methods of administration of a peptidic compound of the invention through the skin or mucosa include, but are not necessarily limited to, topical application of a suitable pharmaceutical preparation, transdermal transmission, injection and epidermal administration. Also contemplated for delivery of a peptidic compound of the invention is a patch containing therein a peptidic compound of the invention. A patch can be applied to the skin, or to other tissue, e.g., gum tissue. Any known patch delivery system that is suitable for oral delivery system can be used. See, e.g., U.S. Pat. No. 6,146,655.

As one of the specific embodiment of this invention, saline formulation of AC-100 can be directly applied to the surface of the exposed pulp in a dental defect before the defect is sealed with any type of undegradable sealing materials. Even in the event that the defect is not as deep as to reach the pulp, the saline formulation of AC-100 can be directly applied to the surface of the dentin in the defect before it is sealed. As proven in the Example of the invention, by addition of this simple step to the current standard treatment procedure of dental cavity, the pulp tissue is well protected from inflammation that is typical to dental defects and often worsened by the chemicals derived from the sealing materials, and thereby reduces the irritation and pain which is usually experienced by the subjects within hours or a few days after the defect is sealed. Further, application of AC-100 formulation in such a manner protects the pulp cells from apoptotic or necrotic cell death, which retains high dental viability and assists regeneration of new dentin covering and protecting the pulp. As described in the prior arts, AC-100 is already known to promote proliferation of dental pulp cells in vitro. Therefore, one can expect that AC-100 application to the exposed pulp may help dentinogenesis, which was actually proven in the Example of this invention. However, it was surprisingly demonstrated in this invention that simple application of AC-100 saline formulation protects the pulp and pulp cells and provided significant clinical benefits in the dental defect treatment practice. This method can be combined with any dental sealing materials including, but not limited to, biological materials, polymer resins, metals, alloys, calcium salts, and others. As examples of such dental sealing materials, dentin slice, cotton, collagen sponge (Sulzer Dental, etc.), Vitrebond (3M/ESPE), Single Bond (3M/ESPE), Clearfil (Kuraray), Tetric (Vivadent), and Filtek Z250 (3M/ESPE) were shown to be usable in combination with or as a carrier material of AC-100 for the methods described herein.

Peptides of the invention can also be delivered to an individual by administering to the individual a nucleic acid molecule comprising a nucleotide sequence that encodes a peptide of the invention. The terms “polynucleotide” and “nucleic acid molecule” are used interchangeably herein to refer to polymeric forms of nucleotides of any length. The polynucleotides may contain deoxyribonucleotides, ribonucleotides, and/or their analogs. For expression, an expression cassette may be employed. The expression vector will provide a transcriptional and translational initiation region, which may be inducible or constitutive, where the coding region is operably linked under the transcriptional control of the transcriptional initiation region, and a transcriptional and translational termination region. These control regions may be native to a gene encoding the subject peptides, or may be derived from exogenous sources.

Expression vectors generally have convenient restriction sites located near the promoter sequence to provide for the insertion of nucleic acid sequences encoding heterologous proteins. A selectable marker operative in the expression host may be present. Expression vectors may be used for the production of fusion proteins, where the exogenous fusion peptide provides additional functionality, i.e. increased protein synthesis, stability, reactivity with defined antisera, an enzyme marker, e.g. β-galactosidase, etc.

Expression cassettes may be prepared comprising a transcription initiation region, the gene or fragment thereof, and a transcriptional termination region. Vectors include, but are not limited to, plasmids; cosmids; viral vectors; artificial chromosomes (YAC's, BAC's, etc.); mini-chromosomes; and the like. Vectors are amply described in numerous publications well known to those in the art, including, e.g., Short Protocols in Molecular Biology, (1999) F. Ausubel, et al., eds., Wiley & Sons.

Expression vectors may be used to introduce a nucleic acid molecule encoding a subject peptide into a cell of an individual. Such vectors generally have convenient restriction sites located near the promoter sequence to provide for the insertion of nucleic acid sequences. Transcription cassettes may be prepared comprising a transcription initiation region, the target gene or fragment thereof, and a transcriptional termination region. The transcription cassettes may be introduced into a variety of vectors, e.g. plasmid; retrovirus, e.g. lentivirus; adenovirus; and the like, where the vectors are able to transiently or stably be maintained in the cells, usually for a period of at least about one day, more usually for a period of at least about several days to several weeks.

An expression vector comprising a nucleotide sequence encoding a peptide of the invention may be introduced into tissues or host cells by any number of routes, including viral infection, microinjection, or fusion of vesicles. Jet injection may also be used for intramuscular administration, as described by Furth et al. (1992), Anal Biochem 205:365-368. The expression vector may be coated onto gold microparticles, and delivered intradermally by a particle bombardment device, or “gene gun” as described in the literature (see, for example, Tang et al. (1992), Nature 356:152-154), where gold microprojectiles are coated with the expression vector, then bombarded into skin cells.

Dosages

Although the dosage used will vary depending on the clinical goals to be achieved, a suitable dosage range is one which provides up to about 1 μg, to about 1,000 Φg, to about 10,000 μg, to about 25,000 μg or about 50,000 μg of a peptide of the invention. Peptides of the invention can be administered in a single dosage or several smaller dosages over time. In one embodiment the formulation is administered one time and not administered again.

The effect on reduction in inflammation, reduction in pain, reduction in number of necrotic cells, reduction in number of apoptotic cells, or other parameter may be dose-dependent. Therefore, to increase potency by a magnitude of two, each single dose is doubled in concentration. Increased dosages may be needed to achieve the desired therapeutic goal. The invention thus contemplates administration of multiple doses to provide and maintain an effect on bone loss, bone strength, or other parameter. When multiple doses are administered, subsequent doses are administered within about 16 weeks, about 12 weeks, about 8 weeks, about 6 weeks, about 4 weeks, about 2 weeks, about 1 week, about 5 days, about 72 hours, about 48 hours, about 24 hours, about 12 hours, about 8 hours, about 4 hours, or about 2 hours or less of the previous dose.

In view of the teaching provided by this disclosure, those of ordinary skill in the clinical arts will be familiar with, or can readily ascertain, suitable parameters for administration of peptides according to the invention.

Formulations

In general, peptidic compounds are prepared in a pharmaceutically acceptable composition for delivery to a host. Pharmaceutically acceptable carriers preferred for use with the peptidic compounds of the invention may include sterile aqueous of non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. A composition comprising a peptidic compound of the invention may also be lyophilized using means well known in the art, for subsequent reconstitution and use according to the invention. Also of interest are formulations for liposomal delivery, and formulations comprising microencapsulated peptidic compounds. Further, the peptidic compounds of this invention can be formulated with appropriate carrier materials to fill the defective space in or between hard tissues such as dental cavity, pocket between a tooth and alveolar bone, socket after a tooth has fallen off or been extracted, deteriorated alveolar, jaw, or sinus bones, and so forth, wherein the carrier is selected from a group comprising collagen, hyaluronic acid, polylactic acid, polyethylene glycol, hydroxyapatite, tricalcium phosphate, in particular, β-tricalcium phosphate, and resin composite used in filling defects in skeletal tissues.

In general, the pharmaceutical compositions can be prepared in various forms, such as suspensions, salves, lotions and the like. In some embodiments, where delivery of a peptidic compound of the invention is to oral tissues, a peptidic compound of the invention may be formulated in a toothpaste, a mouthwash, or may be coated on or embedded in a dental floss or tooth blush. Pharmaceutical grade organic or inorganic carriers and/or diluents suitable for oral and topical use can be used to make up compositions comprising the therapeutically-active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents. Preservatives and other additives may also be present such as, for example, anti-pathogenic agents (e.g., antimicrobials, antibacterials, antivirals, antifungals, etc.), antioxidants, chelating agents, and inert gases and the like.

A peptidic compound of the invention can be administered with any other known agent that promotes hard tissue formation or prevents the loss of hard tissue. Thus, combination therapy is contemplated. Other agents that can be administered with a peptide of the invention include, but are not limited to, skeletal growth factors such as the family molecules of BMP, TGF, FGF, PDGF, and IGF, and pulp capping agent including various calcium salts such as calcium hydroxide and calcium sulfate. A peptidic compound of the invention can be administered simultaneously with (e.g., in admixture with, or in separate formulations) another agent that reduces bone loss; or can be administered within about 15 minutes, about 30 minutes, about 60 minutes, about 2 hours, about 5 hours, about 10 hours, about 12 hours, about 24 hours, about 36 hours, about 4 days, about 7 days, or more, of another agent that reduces bone loss. In addition, two or more peptidic compounds of the invention can be administered simultaneously or within about 15 minutes, about 30 minutes, about 60 minutes, about 2 hours, about 5 hours, about 10 hours, about 12 hours, about 24 hours, about 36 hours, about 4 days, about 7 days, or more of each other. In a particular embodiment the peptide AC-100 is administered one time and not administered again.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

Deep defects in molars in canines were treated with a formulation containing a peptide of the invention referred to as AC-100. The primary purpose of the study was to evaluate the degree of new dentin regeneration in the defects because AC-100 has been known to possess in vitro activities to promote proliferation of primary human dental pulp cells. A formulation of calcium hydroxide named “Dycal” that is widely used for pulp capping by dentists was employed as a positive control. The teeth treated with Dycal showed modest regeneration of new dentin in the defects and also demonstrated severe inflammatory reactions as well as many apoptotic and necrotic cell death in the pulp cavities. On the other hand, the teeth treated with AC-100 formulation demonstrated almost complete reduction of inflammatory reactions as well as apoptotic or necrotic cell death in the pulp cavities while it showed superior regeneration of new dentin tissue in the cavities. The test clearly demonstrated that protection of the pulp tissues (which is a marrow tissue for a tooth) provide additional effects on hard tissue regeneration to heal the defects.

### Study Protocol Outline

Deep class V preparations were made without penetrating the pulp cavity on the buccal (labial) side of 8 maxillary teeth (2 premolars and 1 molar on each side of the oral cavity in one canine) in 10-month old Beagle dogs. A radiograph was taken to confirm location of the pulp cavity. The cavities (defects) were drilled perpendicular to the tooth axis (pulp cavity). Following confirmation of the defect, the teeth on the left side of treatment group 1 were treated with a different AC-100 dose (2 uL of 0.1 mg/mL; 1 mg/mL; 10 mg/mL and 100 mg/mL solutions). The solution was left in the dry cavity for 2 minutes after which the base of the cavity was covered with Vitrebond and the teeth were sealed with composite. The teeth on the right side of treatment group 1 were treated for 15 sec with Scotchbond Etchant (Single Bond adhesive system), washed with water, air dried and treated with a different AC-100 dose each (2 uL of 0.1 mg/mL; 1 mg/mL; 10 mg/mL and 100 mg/mL solutions. The solution were left in the dry cavity for 2 minutes after which the base of the cavity was covered with Single Bond, cured with light and the teeth were sealed with composite.

The teeth in treatment group 2 were treated with a different AC-100 dose each (2 uL of 0.1 mg/mL; 1 mg/mL; 10 mg/mL and 100 mg/mL solutions). The solution was left in the dry cavity for 2 minutes after which the cavities were filled with cotton and sealed with Cavit. The dogs in Treatment group 2 were re-treated 3× a week for 1 week (the teeth on the left side) or for 2 weeks (the teeth on the right side). For each re-treatment, the temporary Cavit filling was removed, the teeth were re-treated with the different AC-100 does. The solution was left in the dry cavity for 2 minutes after which the cavities were filled with cotton and sealed with Cavit.

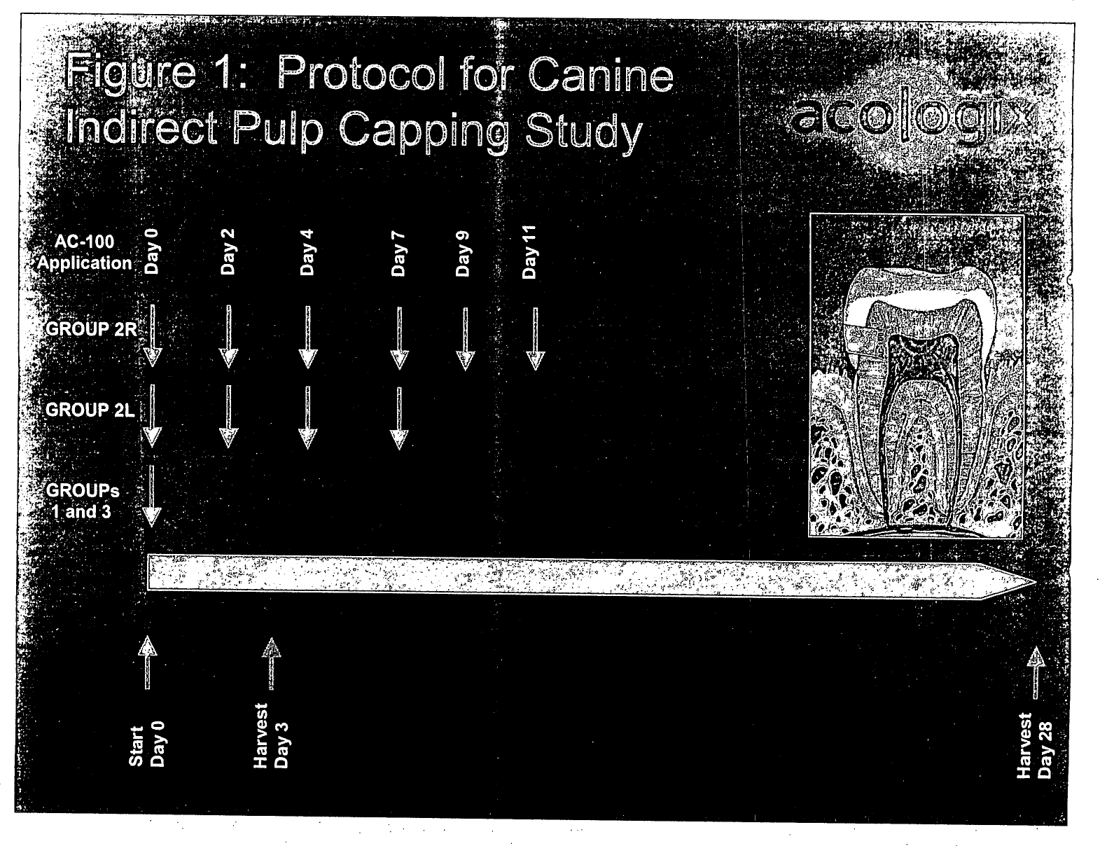

The teeth in treatment group 3 were used as negative (saline) or positive (Dycal) controls. Two of the 4 teeth per animal that were used as negative controls were treated with 2 uL saline. The solution was left in the dry cavity for 2 minutes after which the base of the cavity was covered with Vitrebond and the teeth were sealed with composite. The other 2 teeth used as negative control were treated for 15 sec with Scotchbond Etchant (Single Bond adhesive system), washed with water, air dried and treated with 2 uL saline. The solution was left in the dry cavity for 2 minutes after which the base of the cavity was covered with Single Bond, cured with light and the teeth were sealed with composite. The 2 teeth per animal in treatment group 3 that were used as a positive control were treated with Dycal and sealed with composite using standard operating procedures. The study protocol is exhibited by FIG. 1.

### Treatment

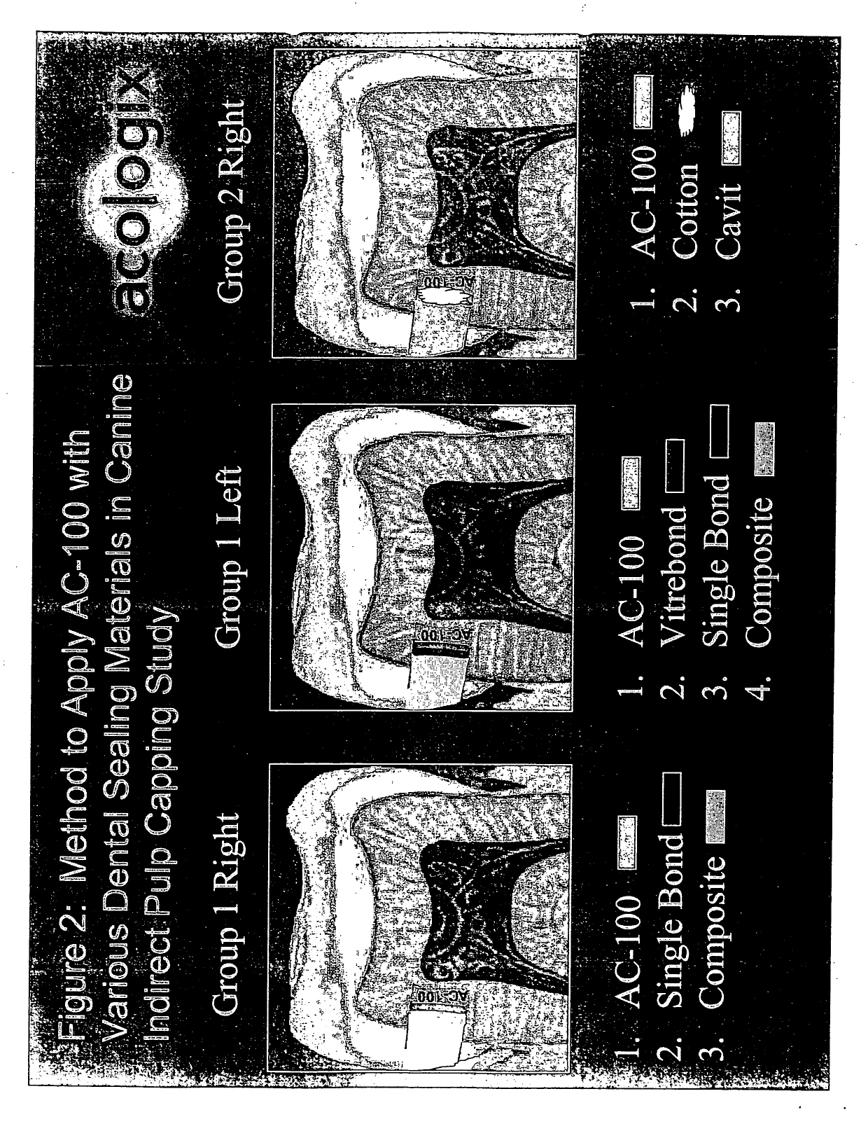

The teeth in respective treatment groups were treated in accordance with the following regimen, which are shown by FIG. 2.

A. Vitrebond and Composite
1. Radiograph upper arcade.
2. Drill cavities with size 8 round bur; undercut cavity with inverted cone (# 34); dry cavity with air.
3. Apply 2 μl AC-100 or saline (negative ctrl) to cavity and allow to stay for 2 min; dry gently with air.
4. Seal with Vitrebond® (brush in as paste, do not compress); light cure-30 sec.
5. Etch using Single Bond® Etchant-15 sec; rinse vigorously with water for 10 seconds; dry cavity with air.
6. Apply Single Bond®; light cure-10 sec.
7. Apply Tetric Flow®; light cure 20 sec; smooth restoration.

B. Single Bond and Composite
1. Radiograph upper arcade.
2. Drill cavities with size 8 round bur; undercut cavity with inverted cone (# 34); dry cavity with air.
3. Apply Scotch Bond® Etchant-15 sec; rinse vigorously with water for 10 seconds; air dry cavity.
4. Apply 2 μl AC-100 or saline (negative ctrl) to cavity and allow to stay for 2 min; dry gently with air.
5. Apply Single Bond®; light cure-10 sec.
6. Apply Tetric Flow®; light cure 20 sec; smooth restoration.

C. Dycal
1. Radiograph upper arcade.
2. Drill cavities with size 8 round bur; undercut cavity with inverted cone (# 34); dry cavity with air.
3. Cover base with Dycal®-allow to set 2 min.
4. Apply Single Bond®; light cure-10 sec.
5. Apply Tetric Flow®; light cure 20 sec; smooth restoration.

D. Multiple Treatments—Cavit
1. Radiograph upper arcade.
2. Drill cavities with size 8 round bur; undercut cavity with inverted cone (# 34); dry cavity with air.
3. Apply 2 μl AC-100 or saline (negative ctrl) to cavity and allow to stay for 2 min; dry gently with air.
4. Apply cotton pellet; seal with Cavit®; smooth temporary restoration.

### Histology

After the specimens were received, the specimens were processed, decalcified and embedded in paraffin. The maxilla specimens were stained with Haematoxylin and eosin (H&E) for new dentin formation evaluation. For immunohistochemistry evaluations, the sections were stained with TUNEL assay for evaluation of apoptosis (score 0-4), and CD45 antibody for inflammation evaluations.

### Histopathology

Histopathological assessment of the stained section was done without knowledge of treatment group. Qualitative observations included the analysis of the tooth and underlying bone, including any soft tissue, which may have come into contact with the test material formulation. Cellular infiltrates (score 0-16) and inflammatory responses (score 0-4) at the repair site and the soft tissues were also evaluated.

### Histomorphometry

Static histomorphometric analysis was performed on sections stained with toluidine blue stain to determine the percent area of dentin in-growth present within the samples using an OsteoMeasure® image analysis system, and associate software (version 4.00c). The dentin in-growth was evaluated by dentin formation score with the range of 0 to 4.

### Results

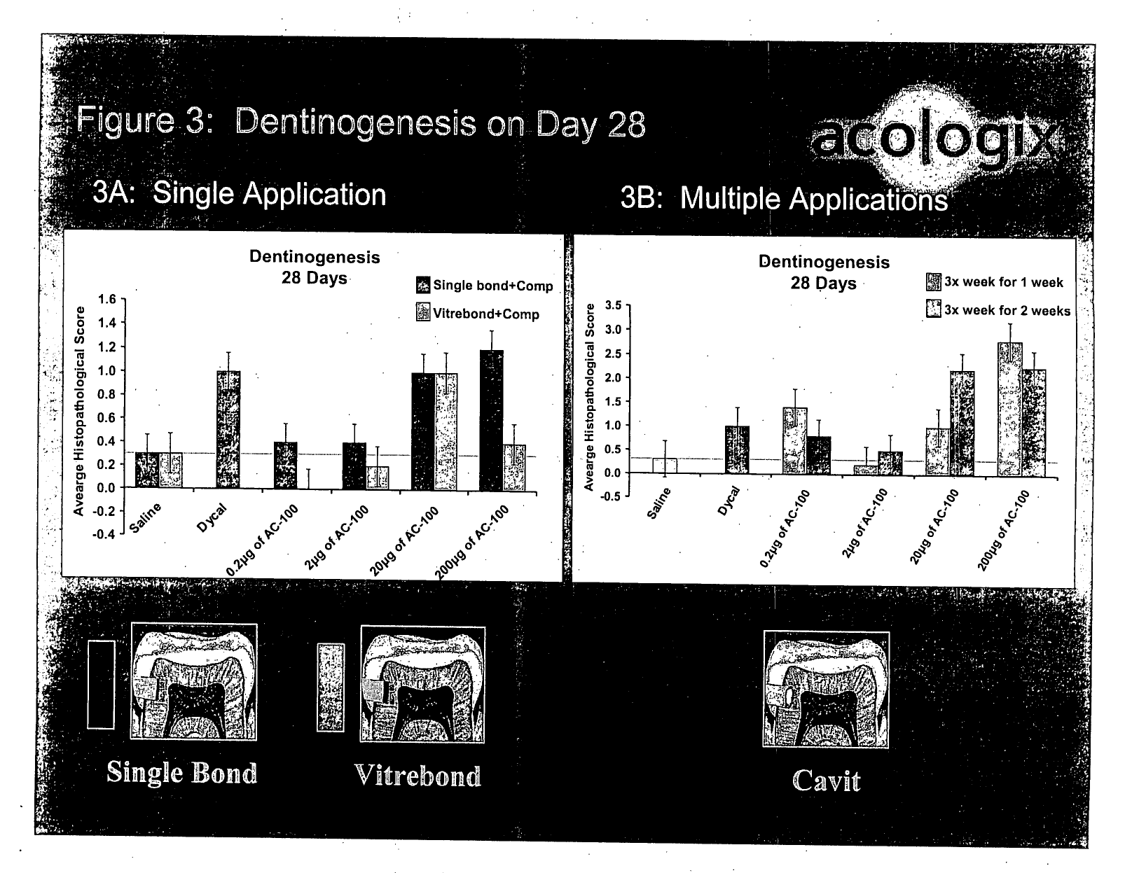

1. At 28 days post surgery the teeth sealed using saline and either Single bond+Composite or Vitrebond+Composite showed minimal new dentin formation. Under these conditions, Dycal, the positive control, showed marginal stimulation of new dentin formation. Application of AC-100 showed a dose dependent stimulation of new dentin formation over the negative control in both the single and the multiple application schedules, with the highest two doses of AC-100 (20 ug and 200 ug) showing results comparable to or better than Dycal, depending on the application schedule (FIG. 3).

2. Within the single application schedule, sealing the teeth with Single Bond+composite (which has demonstrated a somewhat extended release up to 24 hours) worked markedly better than sealing with Vitrebond+Composite at almost all doses of AC-100 used (FIG. 3A).

3. The multiple application schedule worked significantly better than the single application one. At the highest dose of AC-100 used (200 ug) an application schedule of 3 times per week for one week was sufficient to achieve the maximum stimulation of new dentin formation observed in this study, which was 2.8 times better than Dycal and 8 times better than saline (FIG. 3B).

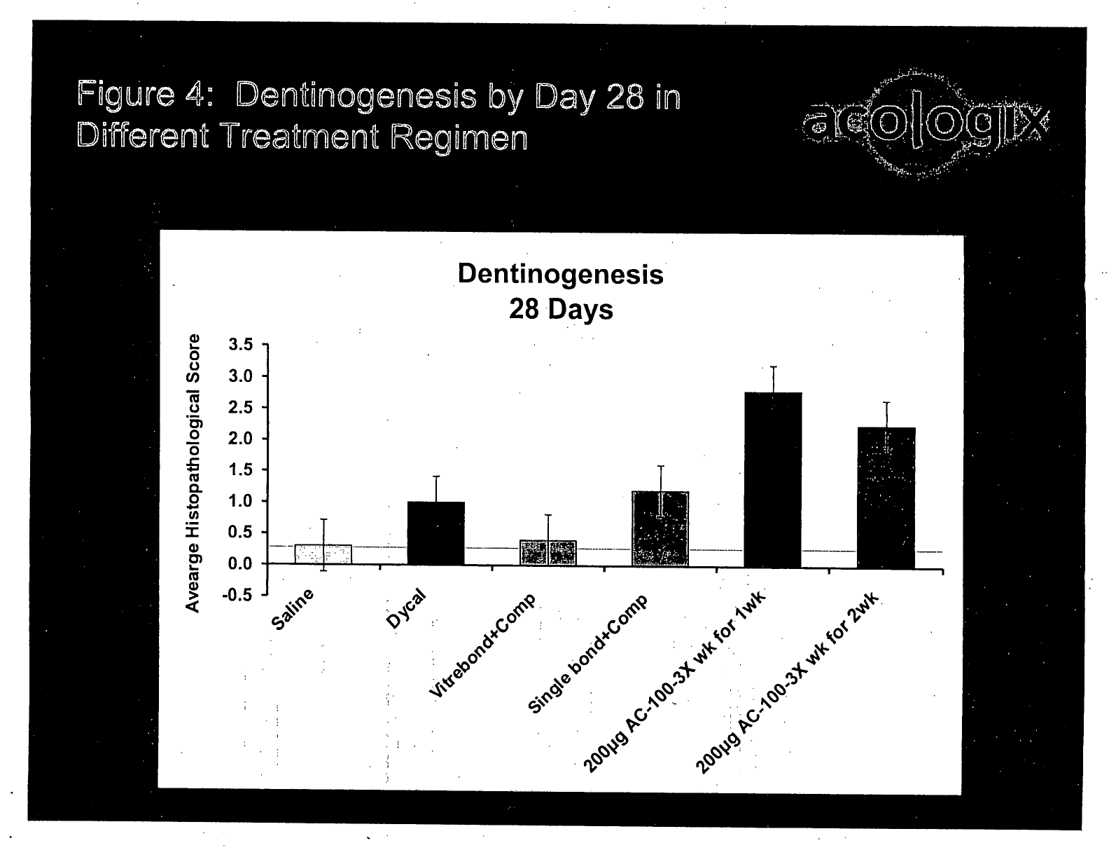

Looking only at the highest dose of AC-100 applied (which showed best results) demonstrates that an extended release of AC-100 will be very beneficial for its activity, especially since the single application, although comparable to Dycal which is the standard of treatment at the moment, showed marginal activity. Moreover, within the single application schedule, sealing the teeth with Single Bond+composite (which has demonstrated a somewhat extended release up to 24 hours) worked markedly better than sealing with Vitrebond+Composite at almost all doses of AC-100 used. Furthermore, at the highest dose of AC-100 used (200 ug), an application schedule of 3 times per week for one week was sufficient to achieve the maximum stimulation of new dentin formation observed in this study, which was 28 times better than Dycal and 8 times better than saline (FIG. 4).

At three days post surgery, the teeth sealed using saline and Single bond+Composite showed a reaction of the pulp tissue to the treatment comprising of both an inflammatory and fibrosis response. Under these conditions, the teeth treated with saline and Vitrebond+Composite or Dycal showed an increased tissue reaction to the treatment. Application of AC-100 showed a dose dependent inhibition of the tissue reaction caused by both Single Bond and Vitrebond, with the highest dose of AC-100 (200 ug) completely abolishing the pulp reaction.

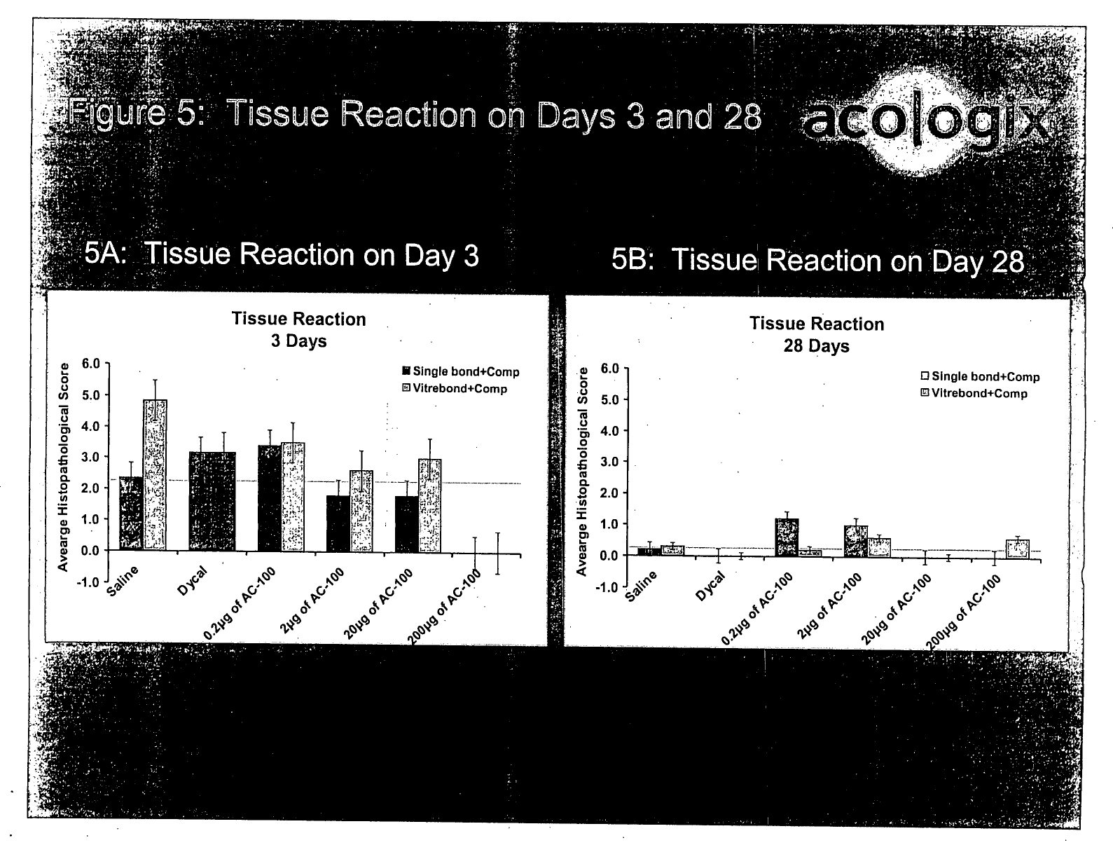

At 28 days post surgery, the pulp tissue response was minimal among all treatments with the main reaction being fibrosis (FIG. 5).

At three days post surgery, the teeth sealed using saline and Single bond+Composite showed an inflammatory response in the pulp tissue. Under these conditions, the teeth treated with saline and Vitrebond+Composite or Dycal showed an increased inflammation. Application of AC-100 completely abolishing the pulp inflammation at almost all doses applied.

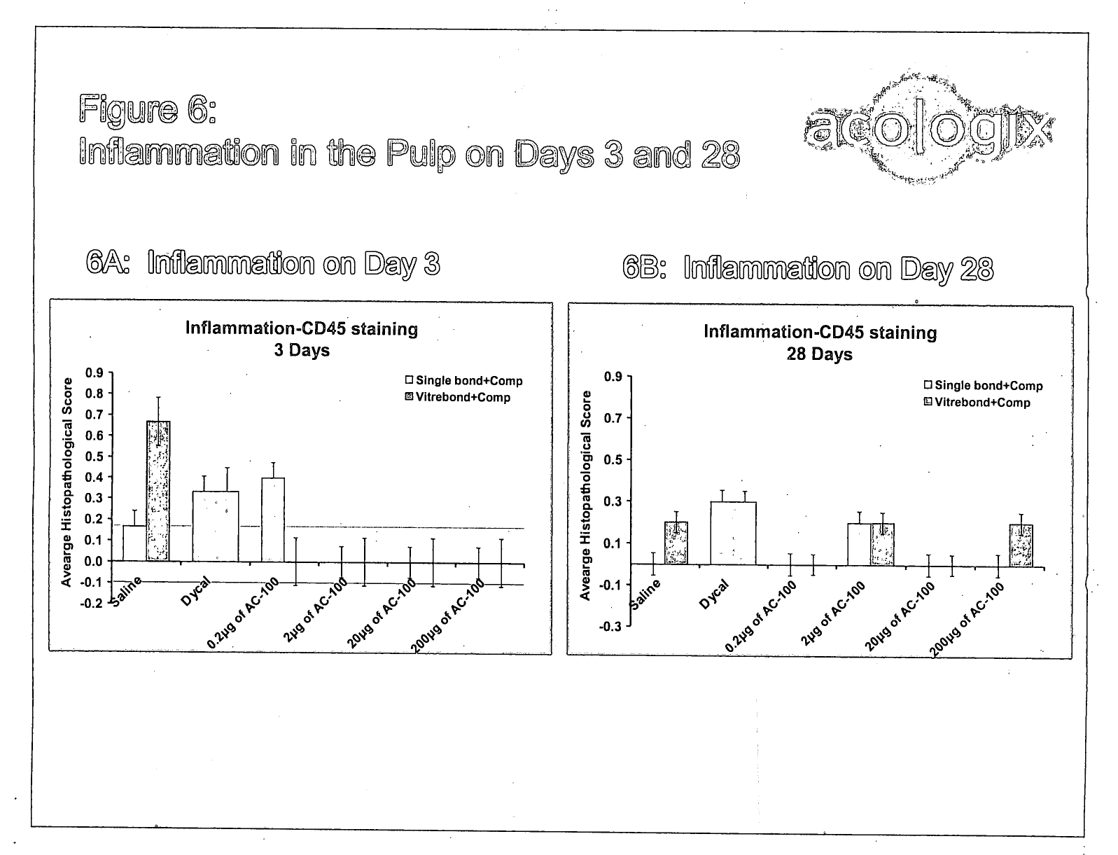

At 28 days post surgery, the pulp inflammation was minimal among all treatments (FIG. 6).

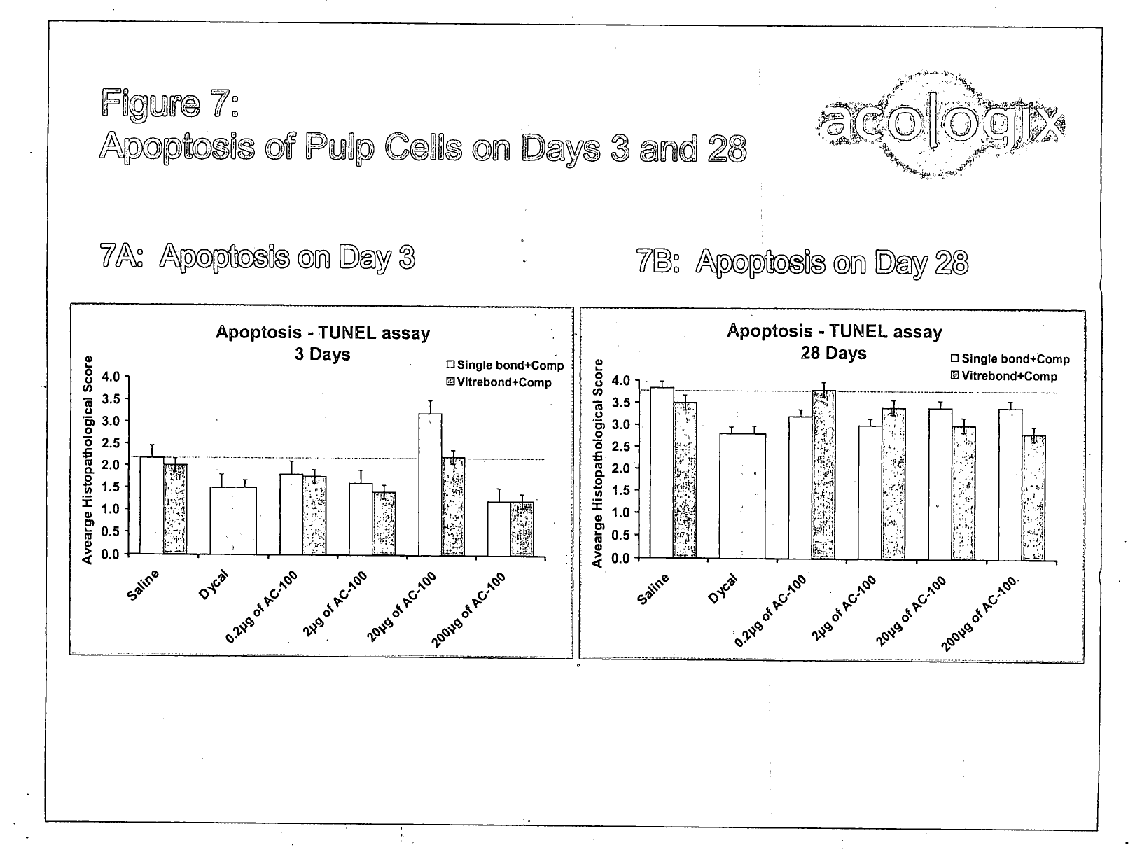

At both 3 and 28 days post surgery application of AC-100 dose dependently decreased the number of apoptotic cells in the pulp in almost all doses applied (FIG. 7).

The dose dependent reduction of the pulp tissue reaction, the number of apoptotic cells and the inflammation by AC-100 at day 3 post surgery demonstrates that AC-100 has beneficial tissue protective properties under these conditions.

In conclusion, AC-100 stimulated new dentin formation in an indirect pulp capping study in dogs in a dose and application method dependent manner. The application of 200 ug AC-100 by three times per week for one week achieved the maximum stimulation of new dentin formation observed in this study, which was 2.8 times better than Dycal and 8 times better than saline. Interestingly, the mechanism of action of AC-100 is significantly different from the mechanism of action of the dentin stimulating agents available for clinical use at present. While the currently approved agents stimulate new dentin formation through non-physiologic irritation of the pulp tissue, leading to localized necrosis and from there to an inflammatory and later reparative response, which stimulates the deposition of new dentin, AC-100 achieves its activity in a tissue protective more physiologic manner by stimulating the existing pulp cells to produce dentin with a reduced inflammatory and apoptotic response.

### Example 2

Sockets after teeth were extracted in canines were treated with a formulation containing a peptide of the invention referred to as AC-100. The objective of the study was to evaluate the new bone regeneration in the defects because anabolic effects of AC-100 have been shown in vitro in primary mesenchimal stem cell culture. Specifically, AC-100 has been shown to dose dependently induce proliferation and differentiation of the cells to osteoblasts. This has been shown in organ cultures of neonatal mouse calvariae and in in vivo injection onto the calvariae of mice. AC-100 has also demonstrated that it stimulates bone fracture healing in rats through a local injection near the fracture site (Lazarov, et. al., ASBMR 2004). Combining these preceding study results and the dentinogenesis activities shown by Example 1 herein, it was intended to evaluate whether or not AC-100 might have beneficial activities in rebuilding a bone bridge over a socket in the alveolar bone after a tooth was extracted in canine.

### Study Protocol Outline

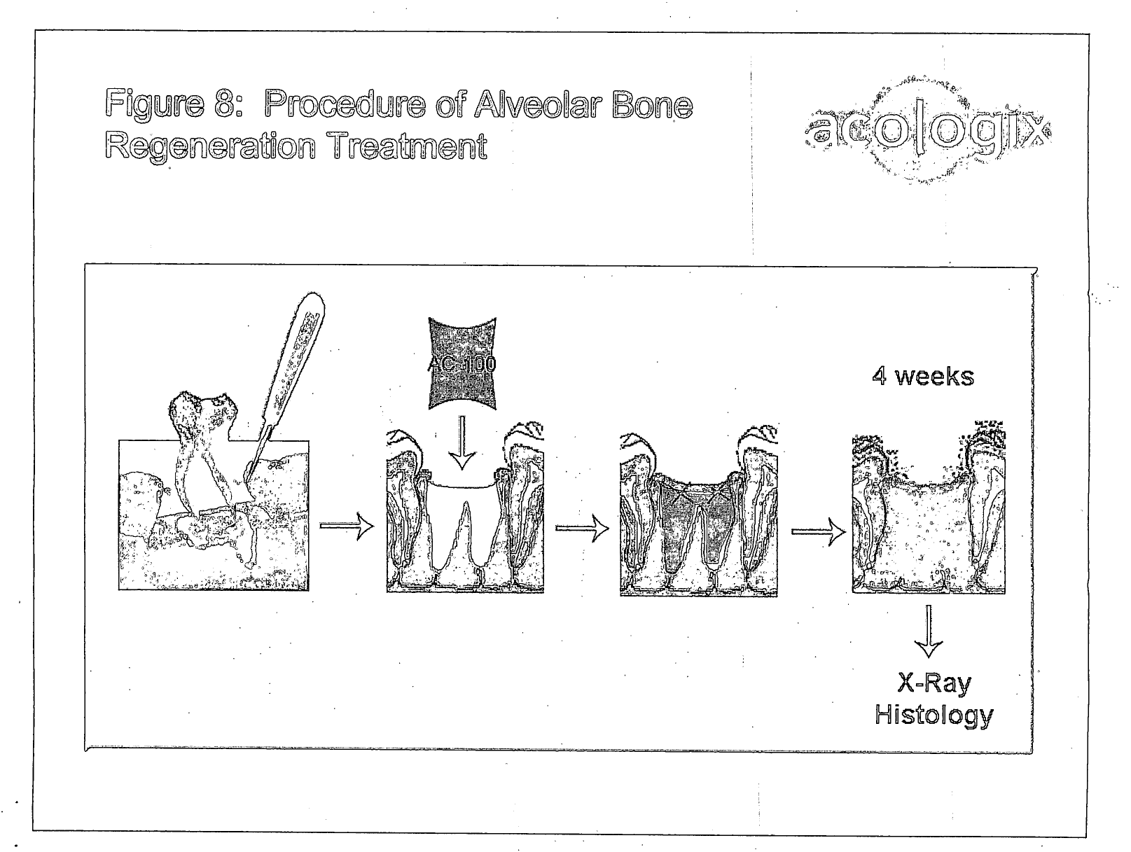

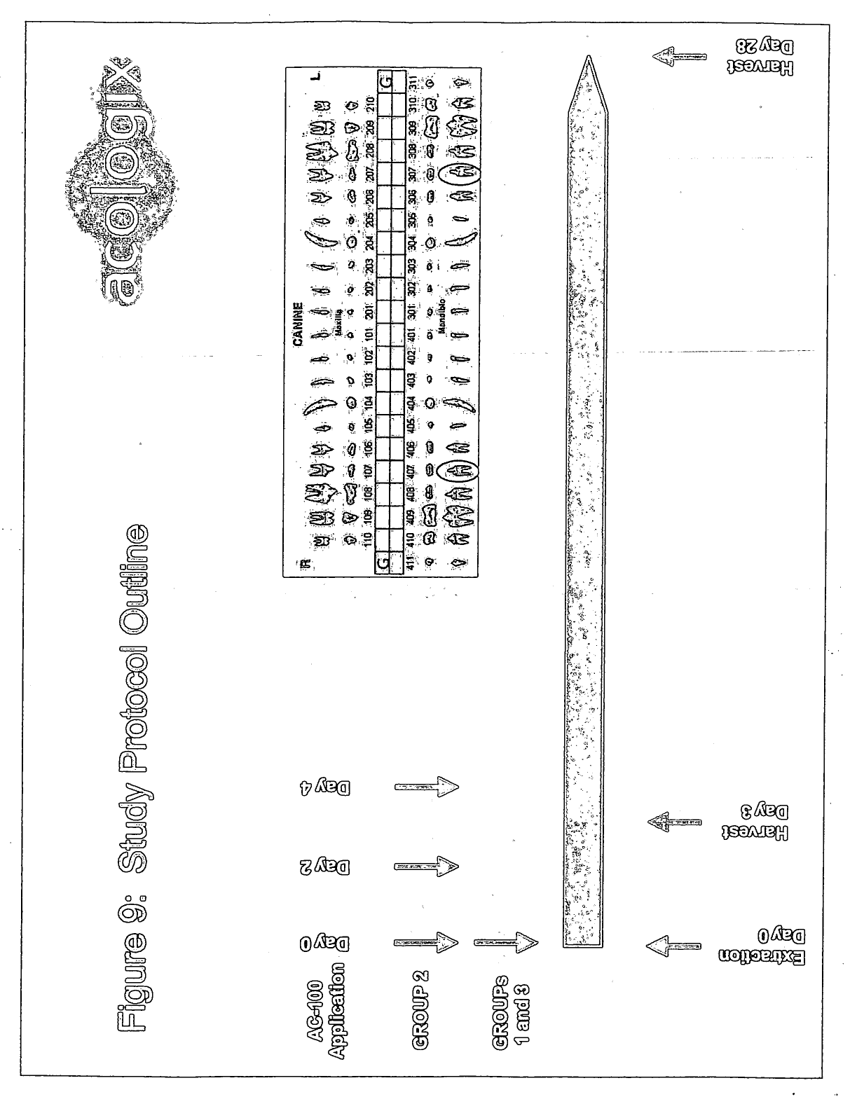

One mandibular premolar per side was extracted. The intra-socket septae in the extraction socket were removed and the wound margins adapted. A collagen sponge soaked in 100 μL of saline, 10 mg/mL AC-100 solution of 100 μL of 100 mg/mL AC-100 solution was applied in the extraction socket. Closure was achieved with interrupted sutures. This procedure from extraction of a tooth to filling the socket is schematically indicated by FIG. 8. A time line and tooth numbering is shown in FIG. 9. For the multiple-treatment group 100 μL of 10 mg/ml or 100 mg/ml AC-100 were injected additionally with

1. Treatment Group I (10 dogs-5 per each time point 3 days and 28 days):
a. Right side: create gingival flap around #407; extract #407 (section with 70 IL); remove intra-socket septa with 55 IL; radiograph; measure socket depth; soak a collagen sponge in 100 μL of 100 mg/ml AC-100; apply collage sponge to socket; close gingival flap with 4-0 Maxon.
b. Left side: create gingival flap around #307; extract #307 (section with 701 L); remove intra-socket septa with 551 L; radiograph; measure socket depth; soak a collagen sponge in 100 μl of 10 mg/ml AC-100; apply collagen sponge to socket; close gingival flap with 4-0 Maxon.

2. Treatment Group 2 (10 dogs-5 per each time point 3 days and 28 days):
a. Right side: create gingival flap around #407; extract #407 (section with 70 IL); remove intra-socket septa with 55 IL; radiograph; measure docket depth; soak a collagen sponge in 100 μL of 100 mg/ml AC-100; apply collagen sponge to socket; close gingival flap4-0 Maxon. These dogs were sedated three times weekly and 100 μL of 100 mg/ml AC-100 were injected in the deepest ⅓ of the socket as determined by initial socket measurement.
b. Left side: create gingival flap around #307; extract #307 (section with 70 IL); remove intra-socket septa with 55 IL; radiograph; measure socket depth; soak a collagen sponge in 100 μL of 10 mg/ml AC-100; apply collagen sponge to socket; close gingival flap with 4-0 Maxon. These dogs were sedated three times weekly and 100 μL of 10 mg/ml AC-100 were injected in the deepest ⅓ of the socket as determined by initial socket measurement.

3. Treatment Group 3 (6 dogs-3 per each time point 3 days and 28 days):
a. Right side: create gingival flap around #407; extract #407 (section with 7011); remove intra-socket septa with 5511; radiograph; measure socket depth; soak a collagen sponge in BMP2; apply collagen sponge to socket; close gingival flap with 4-0 maxon.
b. Left side; create gingival flap around #307; extract #307 (section with 701 l); remove intra-socket septa with 551 l; radiograph; measure socket depth; soak a collagen sponge in 100 μl of saline, apply collagen sponge to socket; close gingival flap with 4-0 maxon.

### Histology

After the specimens were received, the specimens were processed, decalcified and embedded in paraffin. The mandible specimens were stained with Toluidine blue for osteoblast evaluations.

### Histopathology

Histopathological assessment of the stained section was done without knowledge of treatment group. Qualitative observations included the analysis of the underlying bone, including any soft tissue, which may have come into contact with the test material formulation. Fibrous tissue encapsulation, cellular infiltrates and inflammatory responses at the repair site and the soft tissues were also evaluated.

### Histomorphometry

Static histomorphometric analysis was performed on sections stained with toluidine blue stain to determine the percent area of bone in-growth present within the samples using an OsteoMeasure® image analysis system, and associate software (version 4.00c). The amount and quality of the newly formed bone was scored for 0-4, respectively, and evaluated collectively. Tissue reaction such as inflammation and presence of fibrous tissue were also evaluated using the respective scoring system.

### Results

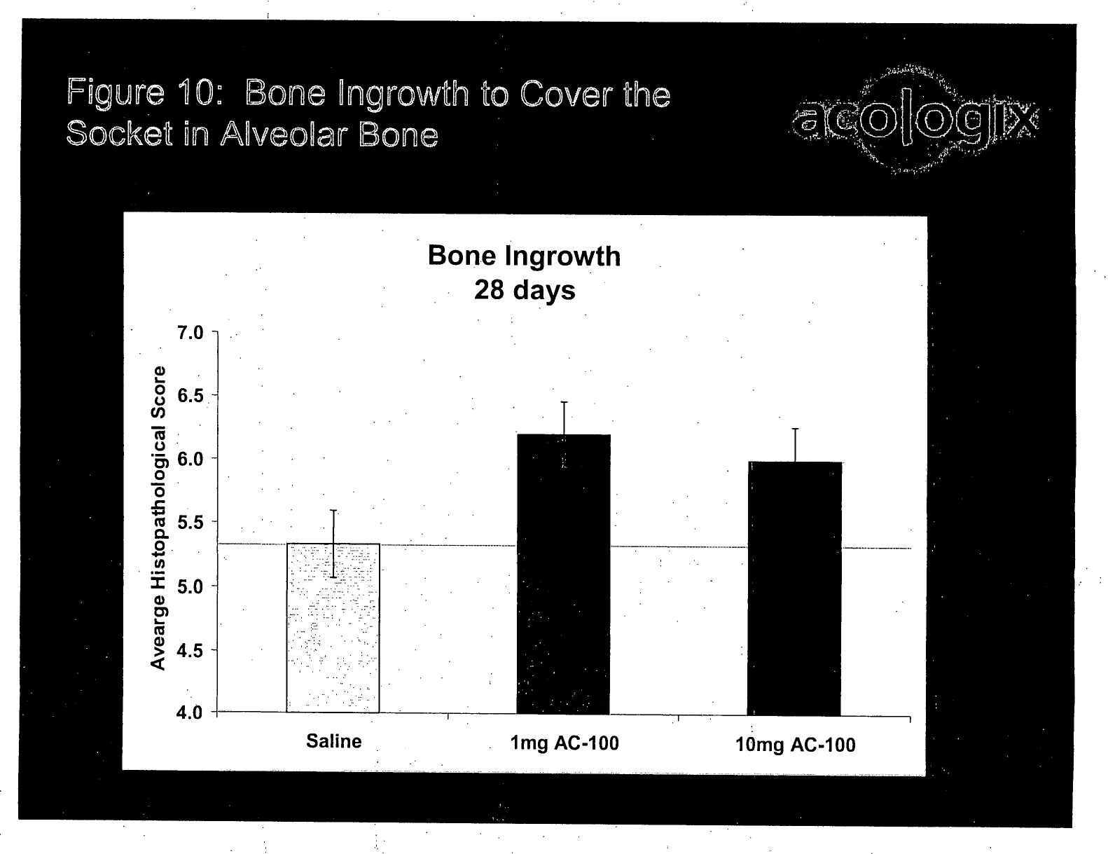

1. A single application at the time of surgery both 1 mg and 10 mg of AC-100 on a collagen sponge caused stimulation of bone growth. A graph of results are shown in FIG. 10.

2. Multiple re-applications of AC-100 directly in the extraction sockets were not as effective as the single application regimen on bone growth.

The bone ingrowth endpoint comprises of two parameters assayed independently -amount of new bone bridging the defect and quality of the new bone (lamellar versus woven bone). The two parameters were separated and it was observed that AC-100 dose dependently increases the quality of the new bone formed in the defect, while at the same time the peptide dose dependently decreases the rate with which the defect is filled with new bone.

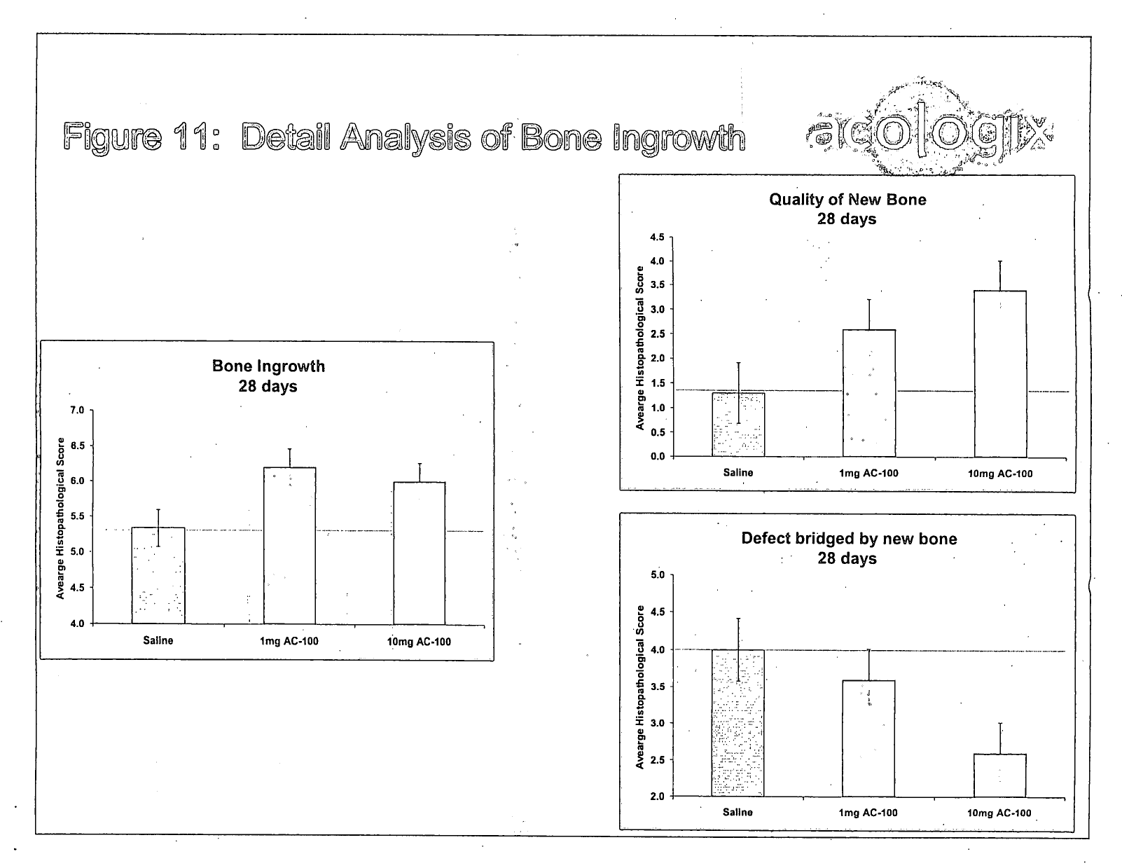

The dose dependent increase in the quality of the new bone formed in the defect cause by DentoninAC-100 can be due to several factors, the most prominent of which are:
1. an increase in the rate of remodeling of the newly formed bone which can account for both the increased bone quality and the decreased rate of defect bridging;
2. an increase in the healing rate after tooth extraction which initiates earlier bone formation and from there gives more time for bone remodeling. Graphs of results are shown in FIG. 11.

Following tooth extraction, remodeling and resportionresorption of the alveolar bone at the extraction site characterize wound healing. The healing of an extraction socket involves a series of events including the formation of a coagulum that is replaced by (i) a provisional connection tissue matrix, (ii) woven bone, and (iii) lamellar bone, and bone marrow. During the healing process a hard tissue bridge—cortical bone—is formed, which “closes” the socket. Thus, the healing of an extraction socket appears to have many features in common with events that characterize new tissue formation in a fracture of a long bone.

The time line of these healing processes in the dog is approximately the following. During the first 3 days of healing, a blood clot is found to occupy most of the extraction site. After seven days this clot is in part replaced with a provisional matrix (PM). On day 14, the tissue of the socket is comprised of PM and woven bone (highly vascularised bone tissue with coarse, undulating, interwoven and randomly oriented collagen fiber bundles and randomly distributed osteocyte lacunae, found in embryonic and fetal bone and fracture callus, normally remodeled and replaced with lamellar bone). On day 30, mineralized woven bone occupies 88% of the socket volume. This tissue decreases to 14% on day 180 and is replaced by lamellar bone (dense bone with fine collagen fibers organized into sheets of a few microns thickness, lamellae, within which the fibers are parallel in orientation). The portion occupied by bone marrow (BM) at day 30 specimens is about 0%, but increase to 85% on day 180.

The two bone ingrowth parameters that were assay at day 28 represent the following:

1. Quality of new bone represents the stage of maturity that the new bone has reached at this time point (woven bone, being the immature and lamellar bone plus bone marrow being the mature state).

2. Defect bridged by new bone represents the extent to which the formed hard tissue bridge “closes” the socket.

As we discussed earlier the dose dependent increase in the quality of the new bone formed in the defect caused by AC-100 can be due in part to an increase in the healing rate after tooth extraction which initiates earlier bone formation and from there gives more time for bone remodeling. To check this hypothesis we looked at the effect that AC-100 has on wound healing at an early time point-3 days.

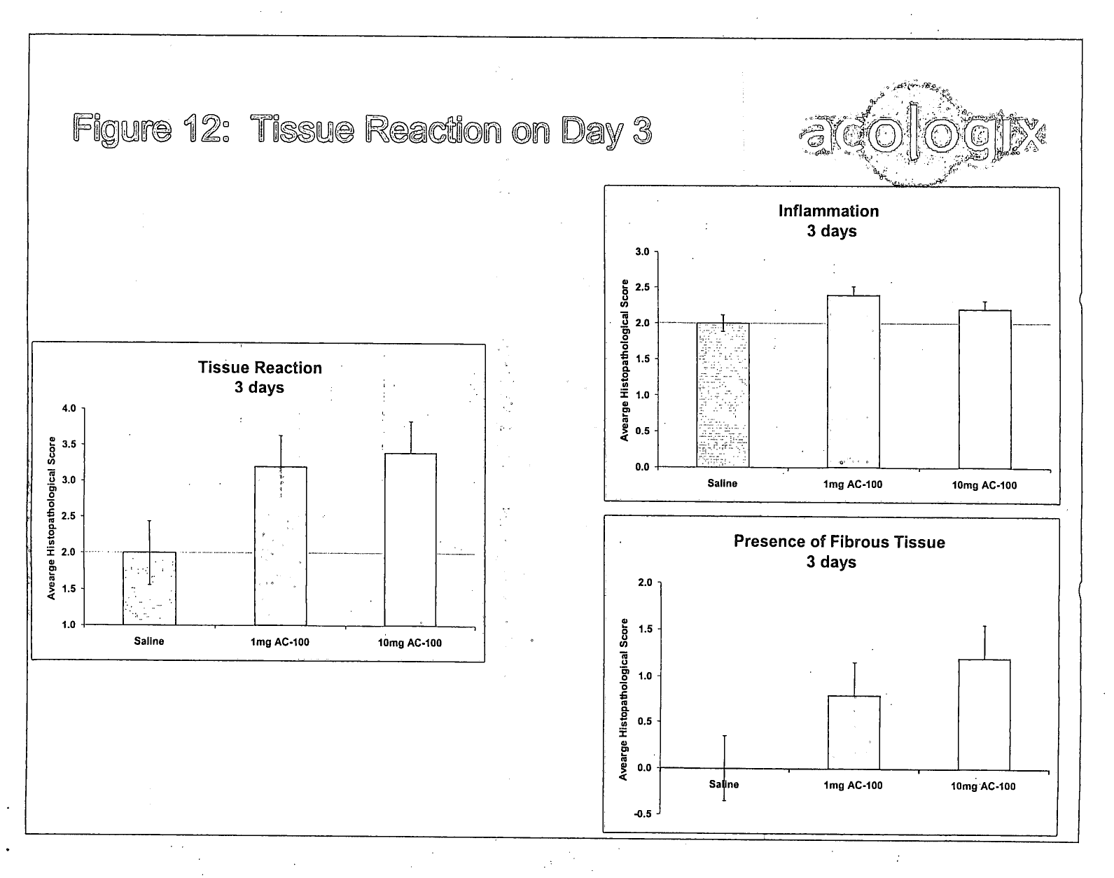

On day 3 after the extraction a dose dependent increase in tissue reaction in the extraction sockets treated with AC-100 was observed (FIG. 12). This was due mainly to an increased presence of connective tissue in the defect and not to an increase in the inflammatory response in the wound. Since during the first 3 days of healing, a blood clot is expected to occupy most of the extraction site (as is the case in the saline control) and its replacement with a provisional matrix (PM) is not expected until day 7, the dose dependent increase in the formation of connection tissue and PM bridging the defect is an indication of an increased rate of wound healing in the early stages of the defect healing.

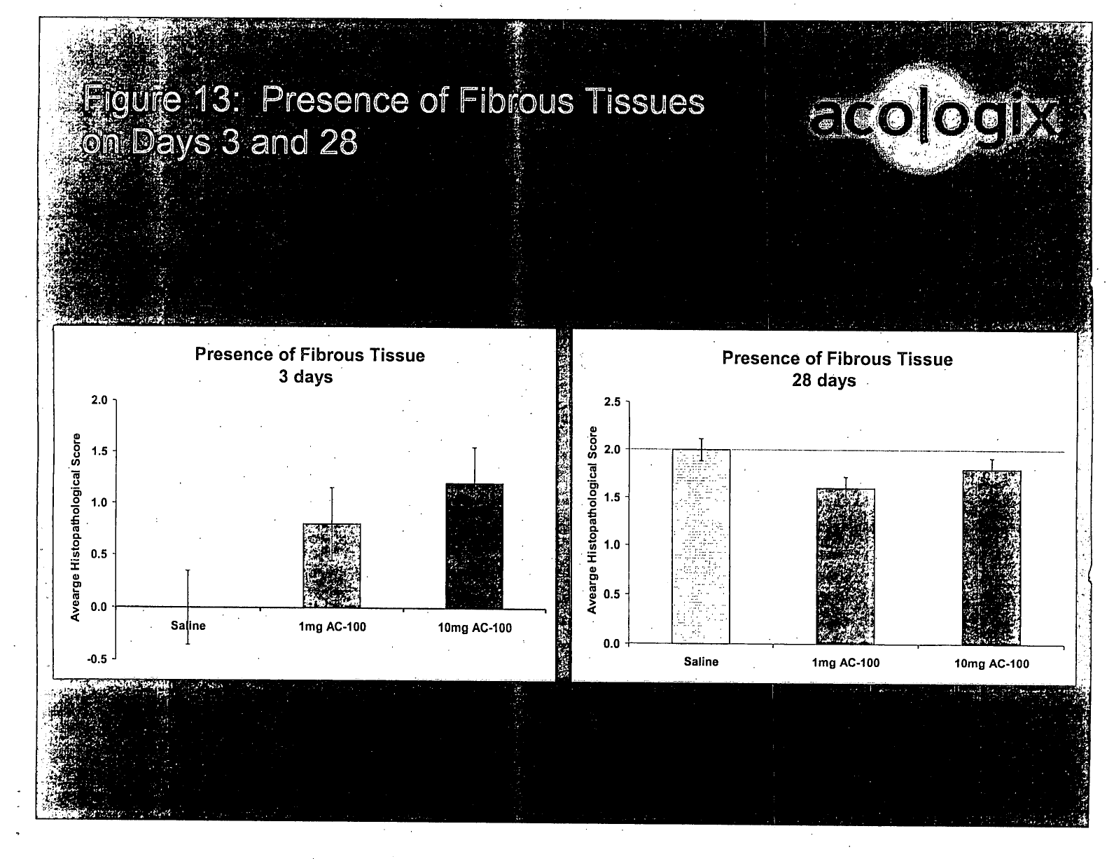

Following further the fate of the increased connection tissue and provisional matrix in the defect we wanted to confirm whether it matures to bone (positive effect) or forms fibrous scar tissue (negative effect). Thus, we compared the fibrous tissue presence in the defects treated with AC-100 or saline control on both days 3 and 28 (see FIG. 13). At day 3 after the extraction, we observed a dose dependent increase in the presence of connective tissue and provisional matrix in the defects treated with AC-100. However, by day 28, the percentage of connective tissue in the defects treated with AC-100 and the defects treated with saline was comparable, thus indicating that the AC-100 induced connective tissue and provisional matrix mature to form new bone and not fibrous scar tissue.

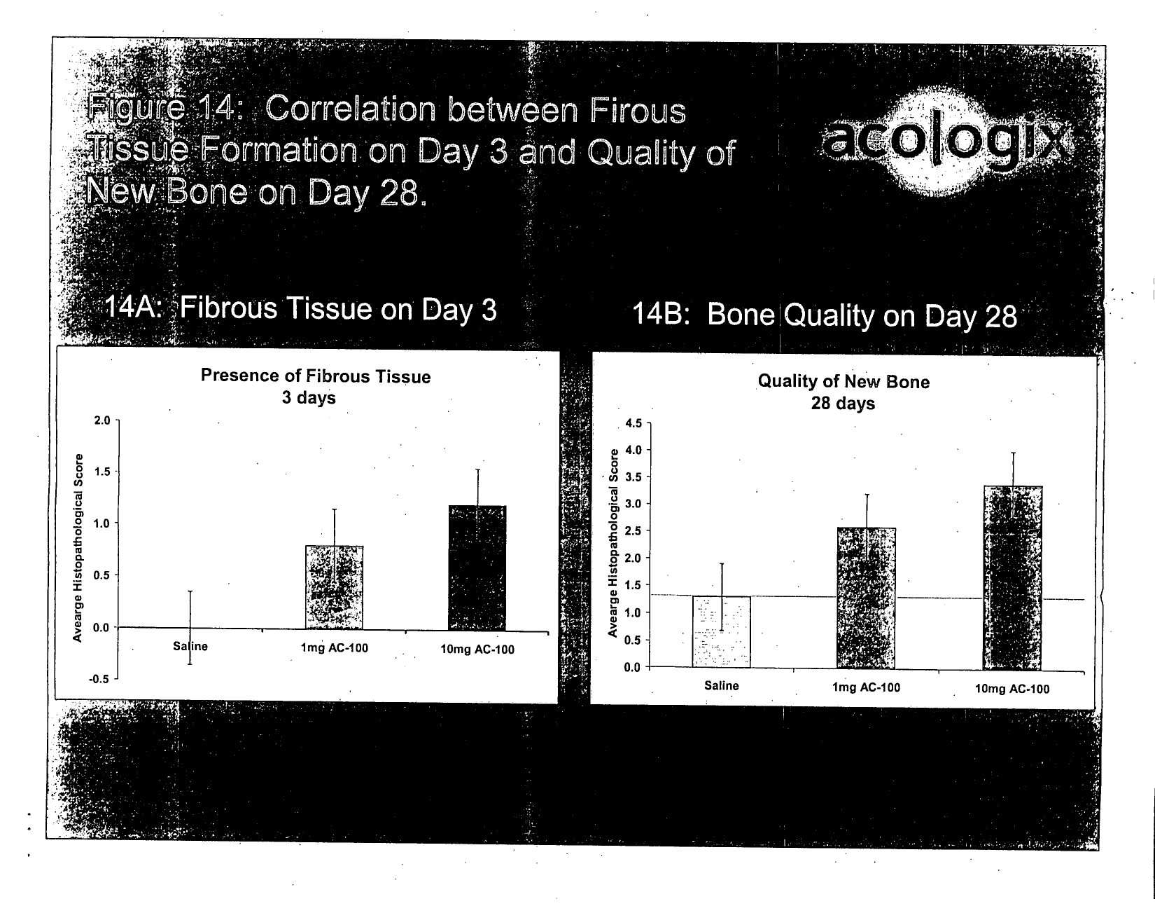

Consequently the observation that AC-100 stimulates an increase in the healing rate after tooth extraction and from there an earlier bone formation and remodeling, can explain the dose dependent increase in the quality of the new bone formed in the defects treated with AC-100. See FIG. 14. However, it does not preclude the possibility that an increase in the rate of remodeling of the newly formed bone can also play a role in the effects of AC-100 on alveolar bone regeneration.

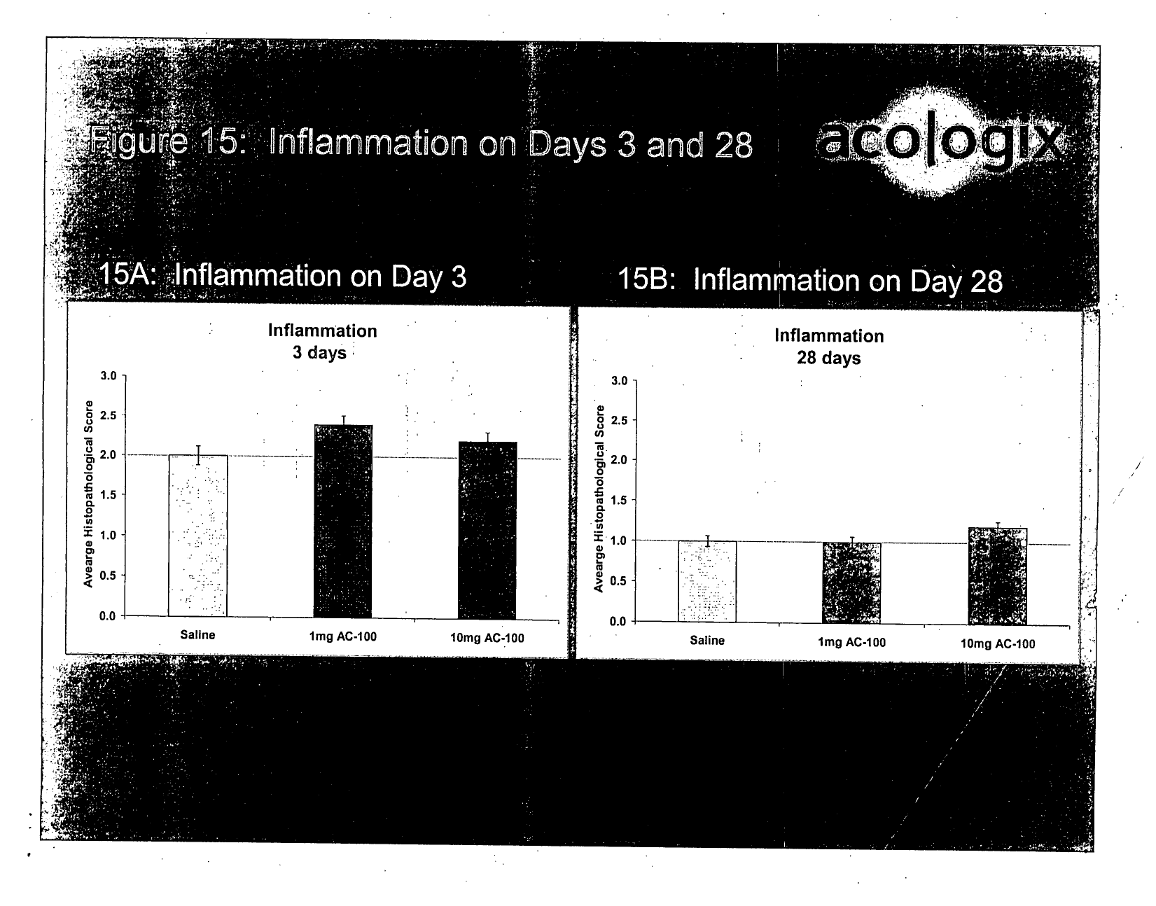

Both at day 3 and at day 29 there was no significant difference in the inflammatory response between the defects treated with AC-100 or saline control (see FIG. 15).

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of treating a skeletal tissue defect, comprising:
identifying an area of a skeletal tissue defect in a patient; and
administering to the patient an amount of a peptide compound sufficient to reduce inflammation in the area of the skeletal tissue defect, wherein the peptide compound comprises about 15 to about 28 amino acids in a sequence, wherein the peptide compound enhances bone growth, wherein each amino acid may be in D- or L-conformation, and wherein the sequence comprises a binding motif chosen from an integrin binding motif, a glycosaminoglycan binding motif, and a calcium binding motif.

2. The method of claim 1, wherein the skeletal tissue defect is a tooth surface and administering is to a tooth surface area created by drilling at an area of decay on the tooth and the sequence comprises each of an integrin binding motif, a glycosaminoglycan binding motif, and a calcium binding motif.

3. The method of claim 1, wherein the integrin binding motif is an RGD sequence and wherein the glycosaminoglycan motif has the sequence SGDG (SEQ ID NO:14).

4. The method of claim 1, wherein the calcium binding motif has the sequence DXDXSXFXGXXQ (SEQ ID NO:17), wherein X is any amino acid.

5. The method of claim 4, wherein the calcium binding motif has the sequence DNDISPFSGDGQ (SEQ ID NO:18).

6. The method of claim 1, wherein the peptide compound is a peptide having the sequence TDLQERGDNDISPFSGDGQPFKD (SEQ ID NO:13).

7. The method of claim 6, wherein the peptide is in a form of a multimer.

8. The method of claim 7, wherein the multimer is a tandem array of two, three, four, five or six copies of the a peptide having the sequence TDLQERGDNDISPFSGDGQPFKD (SEQ ID NO:13).

9. The method of claim 1, wherein the skeletal defect is bone tissue surrounding a tooth and administering comprises injecting the peptide compound between the tooth and surrounding bone.

10. The method of claim 1, wherein the peptide consists of at least 15 and not more than 28 amino acids.

11. The method of claim 10, wherein the peptide is bound to a biocompatible polymer.

12. The method of claim 11, wherein the biocompatible polymer is chosen from polyglycolide (PGA), poly(DL-lactide) (DL-PLA), poly(DL-lactide-co-glycolide) (DL-PLG), poly(L-lactide) (L-PLA), poly(L-lactide-co-glycolide) (L-PLG), polycaprolactone (PCL), polyethylene glycol (PEG), polydioxanone, a polyesteramide, a copolyoxalate and a polycarbonate.

13. The method of claim 1, wherein the peptide is dispersed in an osteoconductive carrier.

14. The method of claim 13, wherein the carrier is chosen from collagen, hyaluronic acid (HA) and β-tricalcium phosphate (β-TCP).

15. The method of claim 1, wherein the patient is suffering from a disorder chosen from dental caries, periodontal disease, osteoporosis, Paget's disease, osteomalacia, renal osteodystrophy, osteodystrophy resulting from other causes, osteolysis mediated by cancer, fractures, and hyperparathyroidism.

16. The method of claim 13, wherein the skeletal tissue is chosen from alveolar and jaw bone and administering is directly to the skeletal tissue.

17. The method of claim 13, wherein the skeletal tissue is chosen from enamel and dentin.

18. A method of treating a skeletal tissue defect, comprising:
identifying bone marrow cells adjacent to a skeletal tissue defect in a patient; and
administering to the patient an amount of a peptide compound sufficient to reduce bone marrow cell death in the identified area adjacent the skeletal tissue defect, wherein the peptide compound consists of 15 to 28 amino acids in a sequence, wherein the peptide compound enhances bone growth, wherein each amino acid may be in D- or L-conformation, and wherein the sequence comprises a binding motif chosen from an integrin binding motif, a glycosaminoglycan binding motif, and a calcium binding motif.

19. The method of claim 18, wherein the bone marrow cells are adjacent an extracted tooth and the sequence comprises each of an integrin binding motif, a glycosaminoglycan binding motif, and a calcium binding motif.

20. A formulation, comprising:
an osteoconductive carrier; and
a peptide compound consisting of 15 to 28 amino acids in a sequence, wherein the peptide compound enhances bone growth, wherein each amino acid may be in D- or L-conformation, and wherein the sequence comprises a binding motif chosen from an integrin binding motif, a glycosaminoglycan binding motif, and a calcium binding motif.

21. The formulation of claim 20, wherein the peptide is bound to a biocompatible polymer.

22. The formulation of claim 21, wherein the biocompatible polymer is chosen from polyglycolide (PGA), poly(DL-lactide) (DL-PLA), poly(DL-lactide-co-glycolide) (DL-PLG), poly(L-lactide) (L-PLA), poly(L-lactide-co-glycolide) (L-PLG), polycaprolactone (PCL), polyethylene glycol (PEG), polydioxanone, a polyesteramide, a copolyoxalate and a polycarbonate.

23. The formulation of claim 20, wherein the peptide is a peptide having the sequence TDLQERGDNDISPFSGDGQPFKD (SEQ ID NO:13).

24. The formulation of claim 23, wherein the carrier is chosen from collagen, hyaluronic acid (HA) and β-tricalcium phosphate (β-TCP).
